(19) United States
(12) Patent Application Publication
(10) Pub. No.: US 2024/0076287 A1
(43) Pub. Date: Mar. 7, 2024

(54) SOLID FORMS OF A CDK2 INHIBITOR

(71) Applicant: PFIZER INC., NEW YORK, NY (US)

(72) Inventors: Fengjuan CAO, San Diego, CA (US); Kevin Francis DEBOYACE, Mystic, CT (US); Michael HEBERLEIN, San Diego, CA (US)

(73) Assignee: PFIZER INC., NEW YORK, NY (US)

(21) Appl. No.: 18/268,959

(22) PCT Filed: Dec. 21, 2021
(86) PCT No.: PCT/IB2021/062082

§ 371 Date: Jun. 21, 2023

Related U.S. Application Data
(60) Provisional application No. 63/130,530, filed on Dec. 24, 2020.

(51) Int. Cl. C07D 403/12

(52) U.S. Cl. CPC C07D 403/12

(57) ABSTRACT

The invention relates to solid forms of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, to pharmaceutical compositions comprising such solid forms, and to use of such solid forms and pharmaceutical compositions for the treatment of cancer.

### BACKGROUND OF THE INVENTION

### Field of the Invention

This invention relates to solid forms of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (also referred to herein as PF-07104091), to pharmaceutical compositions comprising such solid forms, and to methods of using such solid forms and pharmaceutical compositions for the treatment of cancer.

### Description of Related Art

The compound (1R, 3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091) is a potent inhibitor of cyclin dependent kinase 2 (CDK2), having the structure:

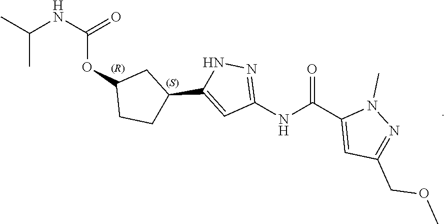

Preparation of PF-07104091, isolated as a crystalline monohydrate (Form 1), is disclosed in International Patent Publication No. WO2020/157652 and in U.S. Pat. No. 11,014,911, the contents of each which are incorporated herein by reference in their entirety.

The present invention provides crystalline forms of PF-07104091 having desirable properties, such as high crystallinity, high purity, low hygroscopicity, favorable dissolution or mechanical properties, improved manufacturability or filterability, and/or favorable stability. The present invention also provides amorphous PF-07104091.

### BRIEF SUMMARY OF THE INVENTION

The present invention provides solid forms of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091).

In some aspects and embodiments, the invention provides a crystalline form of PF-07104091. In some aspects and embodiments, the crystalline form is anhydrous crystalline PF-07104091 (Form 2). In preferred aspects and embodiments, the crystalline form is crystalline PF-07104091 monohydrate (Form 3). In other aspects and embodiments, the crystalline form is anhydrous crystalline PF-07104091 (Form 5).

In other aspects and embodiments, the invention provides an amorphous form of PF-07104091. In some aspects and embodiments, the amorphous form is amorphous PF-07104091 (Form 4).

In one aspect, the invention provides anhydrous crystalline PF-07104091 (Form 2) having:
(1) a powder X-ray diffraction (PXRD) pattern (2θ) comprising: (a) one, two, three, four, five, or more than five peaks selected from the group consisting of the peaks in Table 1 in °2θ±0.2 °2θ; or (b) peaks at 2θ values essentially the same as in FIG. 2;
(2) a Raman spectrum comprising: (a) one, two, three, four, five, or more than five wavenumber (cm⁻¹) values selected from the group consisting of the values in Table 2 in cm⁻¹±2 cm⁻¹; or (b) wavenumber (cm⁻¹) values essentially the same as in FIG. 7; or
(3) a ¹³C solid state NMR spectrum (ppm) comprising: (a) one, two, three, four, five, or more than five resonance (ppm) values selected from the group consisting of the values in Table 3 in ppm±0.2 ppm; or (b) resonance (ppm) values essentially the same as in FIG. 11;
or any combination of two or more of (1)(a)-(b), (2)(a)-(b), and (3)(a)-(b), provided they are not inconsistent with each other.

In a further aspect, the invention provides anhydrous crystalline PF-07104091 (Form 2), having:
(a) a powder X-ray diffraction (PXRD) pattern comprising peaks at 2θ values of: 9.8, 13.3 and 17.4 °2θ±0.2 °2θ;
(b) a Raman spectrum comprising wavenumber (cm⁻¹) values of: 1691, 1582 and 996 cm⁻¹±2 cm⁻¹; or
(c) a ¹³C solid state NMR spectrum comprising resonance (ppm) values of: 24.1, 39.8 and 41.6 ppm±0.2 ppm;
or any combination of two or more of (a), (b), and (c).

In some embodiments, the crystalline form is substantially pure anhydrous crystalline PF-07104091 (Form 2).

In another aspect, the invention provides a pharmaceutical composition comprising anhydrous crystalline PF-07104091 (Form 2), according to the aspects or embodiments described herein, and a pharmaceutically acceptable carrier or excipient.

In one aspect, the invention provides crystalline PF-07104091 monohydrate (Form 3), having:
(1) a powder X-ray diffraction (PXRD) pattern (20) comprising: (a) one, two, three, four, five, or more than five peaks selected from the group consisting of the peaks in Table 4 in °2θ±0.2 °2θ, or (b) peaks at 2θ values essentially the same as in FIG. 3;
(2) a Raman spectrum comprising: (a) one, two, three, four, five, or more than five wavenumber (cm⁻¹) values selected from the group consisting of the values in Table 5 in cm⁻¹±2 cm⁻¹; or (b) wavenumber (cm⁻¹) values essentially the same as in FIG. 8; or
(3) a ¹³C solid state NMR spectrum (ppm) comprising: (a) one, two, three, four, five, or more than five resonance (ppm) values selected from the group consisting of the values in Table 6 in ppm±0.2 ppm; or (b) resonance (ppm) values essentially the same as in FIG. 12; or
or any combination of two or more of (1)(a)-(b), (2)(a)-(b), and (3)(a)-(b), provided they are not inconsistent with each other.

In a further aspect, the invention provides crystalline PF-07104091 monohydrate (Form 3), having:
(a) a powder X-ray diffraction (PXRD) pattern comprising peaks at 2θ values of: 8.4, 10.1 and 21.5 °2θ±0.2 °2θ;
(b) a Raman spectrum comprising wavenumber (cm⁻¹) values of: 1657, 1595 and 1408 cm⁻¹±2 cm⁻¹; or
(c) a ¹³C solid state NMR spectrum comprising resonance (ppm) values of: 25.2, 37.5 and 159.3 ppm±0.2 ppm;
or any combination of two or more of (a), (b), and (c).

In some embodiments, the crystalline form is substantially pure crystalline PF-07104091 monohydrate (Form 3).

In another aspect, the invention provides a pharmaceutical composition comprising crystalline PF-07104091 monohydrate (Form 3), according to the aspects or embodiments described herein, and a pharmaceutically acceptable carrier or excipient.

### BRIEF DESCRIPTION OF THE SEVERAL VIEWS OF THE DRAWINGS

FIG. 1. PXRD pattern of PF-07104091 monohydrate (Form 1).

FIG. 2. PXRD pattern of PF-07104091 (Form 2).

FIG. 3. PXRD pattern of PF-07104091 monohydrate (Form 3).

FIG. 4. PXRD pattern of PF-07104091 (Form 4).

FIG. 5. PXRD pattern of PF-07104091 (Form 5).

FIG. 6. FT-Raman spectrum of PF-07104091 monohydrate (Form 1).

FIG. 7. FT-Raman spectrum of PF-07104091 (Form 2).

FIG. 8. FT-Raman spectrum of PF-07104091 monohydrate (Form 3).

FIG. 9. FT-Raman spectrum of PF-07104091 (Form 5).

FIG. 10. Carbon CPMAS spectrum of PF-07104091 monohydrate (Form 1) (# indicates spinning sidebands).

FIG. 11. Carbon CPMAS spectrum of PF-07104091 (Form 2) (# indicates spinning sidebands).

FIG. 12. Carbon CPMAS spectrum of PF-07104091 monohydrate (Form 3) (# indicates spinning sidebands).

FIG. 13. Carbon CPMAS spectrum of PF-07104091 (Form 5) (# indicates spinning sidebands).

FIG. 14. Differential scanning calorimetry thermogram of PF-07104091 (Form 4) at a ramp rate of 10° C./min.

FIG. 15. Thermogravimetric analysis of PF-07104091 (Form 5).

FIG. 16. Single crystal structure of PF-07104091 monohydrate (Form 3).

### DETAILED DESCRIPTION OF THE INVENTION

The present invention may be understood more readily by reference to the following detailed description of the embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The term “about” means having a value falling within an accepted standard of error of the mean, when considered by one of ordinary skill in the art.

The term “amorphous” as used herein, refers to a solid substance which (1) lacks order in three dimensions, or (2) exhibits order in less than three dimensions, order only over short distances (e.g., less than 10 Å), or both. Amorphous solids give diffuse PXRD patterns typically comprising one or two broad peaks.

The term “anhydrous” as used herein, refers to a crystalline form that only contains the active pharmaceutical ingredient (API) as part of its crystalline lattice.

The term “crystalline” as used herein, means having a regularly repeating arrangement of molecules or external face planes. Crystalline forms may differ with respect to thermodynamic stability, physical parameters, x-ray structure and preparation processes.

The terms “polymorph” or “polymorphic” refers to a crystalline form of a compound with a distinct spatial lattice arrangement as compared to other crystalline forms of the same compound.

The term “solvate” describes a molecular complex comprising a compound (e.g., the active pharmaceutical ingredient (API) of a drug product) and a stoichiometric or non-stoichiometric amount of one or more solvent molecules (e.g., water or ethanol). When the solvent is tightly bound to the compound, the resulting complex will have a well-defined stoichiometry that is independent of humidity. When, however, the solvent is weakly bound, as in channel solvates and hygroscopic compounds, the solvent content will be dependent on humidity and drying conditions. In such cases the complex will often be non-stoichiometric.

The term “hydrate” describes a solvate comprising the compound and a stoichiometric or non-stoichiometric amount of water. A “monohydrate” is a hydrate comprising one molecule of water per molecule of compound (i.e., a 1:1 stoichiometry of water to compound).

The expression “substantially pure” means that the crystalline or amorphous form described as substantially pure comprises less than 5%, preferably less than 3%, and more preferably less than1% by weight of impurities, including any other physical form of the compound (i.e., greater than 95%, preferably greater than 97% and more preferably greater than 99% chemical purity).

As used herein, the term “essentially the same” means that variability typical for a particular method is taken into account. For example, with reference to X-ray diffraction peak positions, the term “essentially the same” means that typical variability in peak position and intensity are taken into account. One skilled in the art will appreciate that the peak positions (2θ) will show some variability, typically as much as ±0.2°. Further, one skilled in the art will appreciate that relative peak intensities will show inter-apparatus variability, as well as variability due to the degree of crystallinity, preferred orientation, prepared sample surface, and other factors known to those skilled in the art and should be taken as qualitative measures only. Similarly, Raman spectrum wavenumber (cm⁻¹) values show variability, typically as much as ±2 cm⁻¹, while ¹³C solid state NMR spectrum (ppm) show variability, typically as much as ±0.2 ppm.

The invention described herein may be suitably practiced in the absence of any element(s) not specifically disclosed herein. Thus, for example, in each instance herein any of the terms “comprising”, “consisting essentially of”, and “consisting of” may be replaced with either of the other two terms.

The solid forms of PF-07104091 described herein may be characterized by any of the following methods: (1) powder X-ray diffraction (PXRD) (2θ); (2) Raman spectroscopy (cm⁻¹); (3) ¹³C solid state NMR spectroscopy (ppm); or (4) differential scanning calorimetry (DSC) (Tg ° C.); or any combination of two or more of methods (1), (2), (3), and (4).

In each of the aspects and embodiments herein that are characterized by PXRD, the PXRD peaks were measured using CuKα radiation at 1.5418λ.

Such solid forms may be further characterized by additional techniques, such as Fourier transform infrared spectroscopy (FTIR), thermogravimetric analysis (TGA), or differential thermal analysis (DTA).

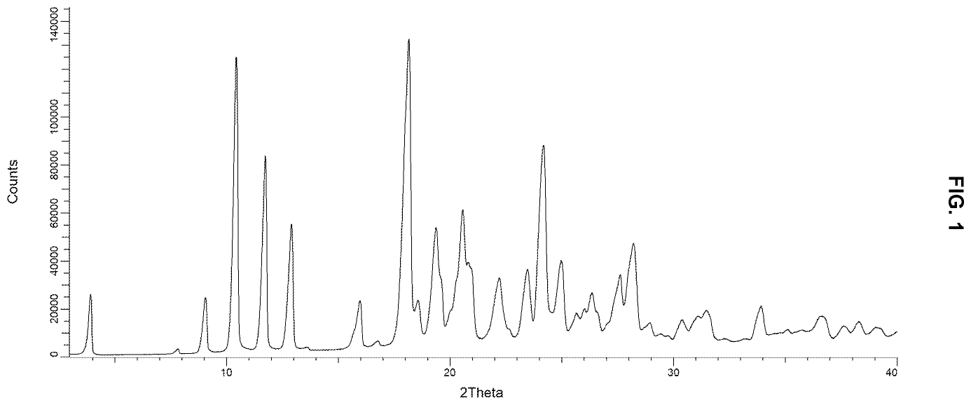

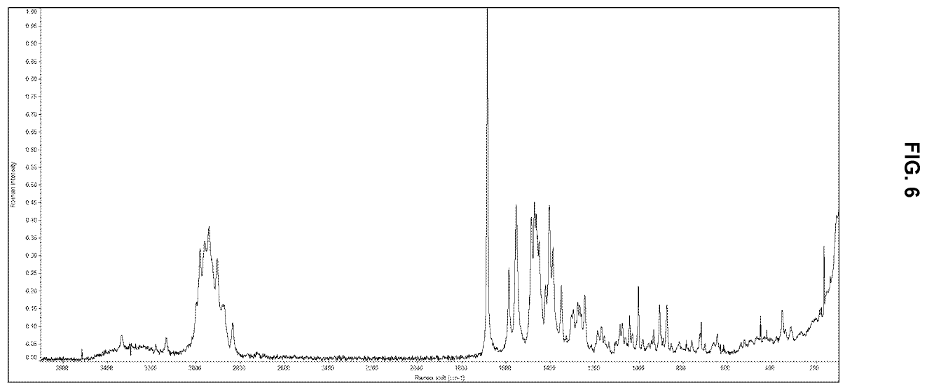

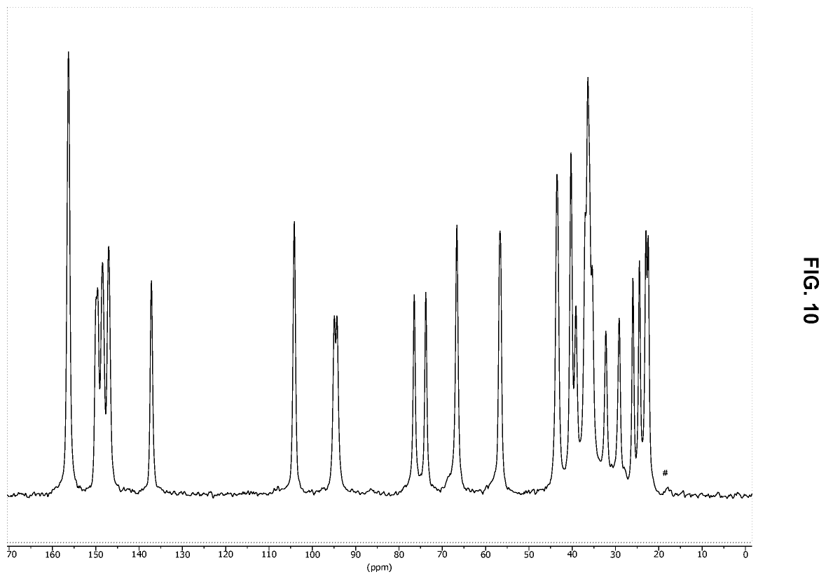

Comparative PXRD, Raman and ¹³C ssNMR data for crystalline PF-07104091 monohydrate (Form 1), described in International Patent Publication No. WO2020/157652 and in U.S. Pat. No. 11,014,911, is provided in FIG. 1, FIG. 6 and FIG. 10, respectively.

In one aspect, the invention provides anhydrous crystalline PF-07104091 (Form 2).

In some embodiments, PF-07104091 (Form 2) is characterized by its powder X-ray diffraction (PXRD) pattern. In other embodiments, PF-07104091 (Form 2) is characterized by its Raman spectrum. In other embodiments, PF-07104091 (Form 2) is characterized by its ¹³C solid state NMR spectrum.

In further embodiments, anhydrous crystalline PF-07104091 (Form 2) is characterized by any combination of two or more of these methods. Exemplary combinations including two or more of the following are provided herein: powder X-ray diffraction (PXRD) pattern (2θ); Raman spectrum wavenumber values (cm⁻¹); or ¹³C solid state NMR spectrum (ppm).

In some embodiments, PF-07104091 (Form 2) is characterized by PXRD and Raman. In other embodiments, PF-07104091 (Form 2) is characterized by PXRD and ¹³C solid state NMR. In other embodiments, PF-07104091 (Form 2) is characterized by Raman and ¹³C solid state NMR. In other embodiments, PF-07104091 (Form 2) is characterized by PXRD, Raman and ¹³C solid state NMR.

In one aspect, the invention provides anhydrous crystalline PF-07104091 (Form 2) characterized by a powder X-ray diffraction (PXRD) pattern.

In one embodiment, the invention provides PF-07104091 (Form 2), having a powder X-ray diffraction (PXRD) pattern comprising peaks at 2θ values of: 9.8, 13.3 and 17.4 °2θ±0.2 °2θ.

In one embodiment, the invention provides PF-07104091 (Form 2), having a powder X-ray diffraction (PXRD) pattern comprising peaks at 2θ values of: 4.2, 9.8, 13.3 and 17.4 °2θ±0.2 °2θ.

In one embodiment, the invention provides PF-07104091 (Form 2), having a powder X-ray diffraction (PXRD) pattern comprising peaks at 2θ values of:7.5, 9.8, 13.3 and 17.4 °2θ±0.2 °2θ.

In another embodiment, the invention provides PF-07104091 (Form 2), having a powder X-ray diffraction (PXRD) pattern comprising peaks at 2θ values of: 4.2, 7.5, 9.8, 13.3 and 17.4 °2θ±0.2 °2θ.

In one embodiment, the invention provides PF-07104091 (Form 2), having a powder X-ray diffraction (PXRD) pattern comprising peaks at 2θ values of: 9.8, 13.3 and 17.4 °2θ±0.2 °2θ; and optionally one or two peaks selected from the group consisting of: 4.2 and 7.5 °2θ±0.2 °2θ.

In another embodiment, the invention provides PF-07104091 (Form 2), having a PXRD pattern comprising three or more peaks at 2θ values selected from the group consisting of: 4.2, 7.5, 9.8, 13.3 and 17.4 °2θ±0.2 °2θ.

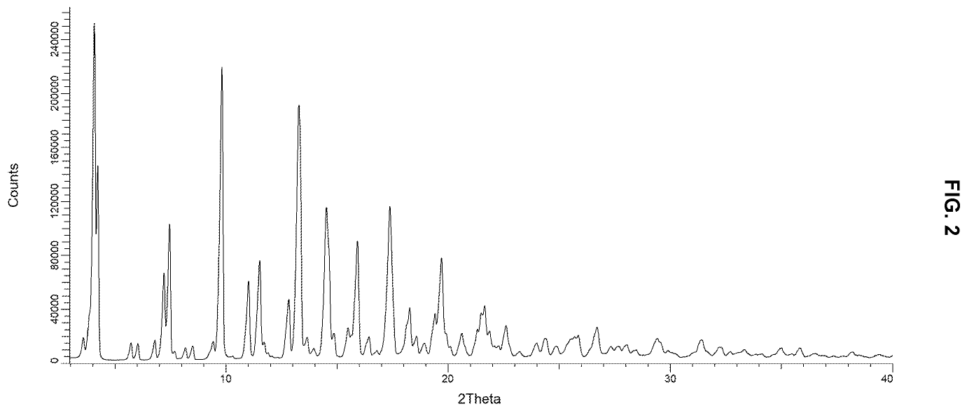

In another embodiment, the invention provides PF-07104091 (Form 2), having a PXRD pattern comprising: (a) one, two, three, four, five, or more than five peaks selected from the group consisting of the peaks in Table 1 in °2θ±0.2 °2θ; or (b) peaks at 2θ values essentially the same as in FIG. 2.

In another aspect, the invention provides anhydrous crystalline PF-07104091 (Form 2) characterized by a Raman spectrum.

In one embodiment, the invention provides PF-07104091 (Form 2), having a Raman spectrum comprising wavenumber (cm⁻¹) values of: 1691, 1582 and 996 cm⁻¹±2 cm⁻¹.

In another embodiment, the invention provides PF-07104091 (Form 2), having a Raman spectrum comprising wavenumber (cm⁻¹) values of: 1691, 1582, 1036 and 996 cm⁻¹+2 cm⁻¹.

In another embodiment, the invention provides a PF-07104091 (Form 2), having a Raman spectrum comprising wavenumber (cm⁻¹) values of: 1691, 1582, 1365 and 996 cm⁻¹+2 cm⁻¹.

In another embodiment, the invention provides PF-07104091 (Form 2), having a Raman spectrum comprising wavenumber (cm⁻¹) values of: 1691, 1582, 1365, 1036 and 996 cm⁻¹+2 cm⁻¹.

In one embodiment, the invention provides PF-07104091 (Form 2), having a Raman spectrum comprising wavenumber (cm⁻¹) values of: 1691, 1582 and 996 cm⁻¹±2 cm⁻¹; and one or two peaks selected from the group consisting of: 1365 and 1036 cm⁻¹±2 cm⁻¹

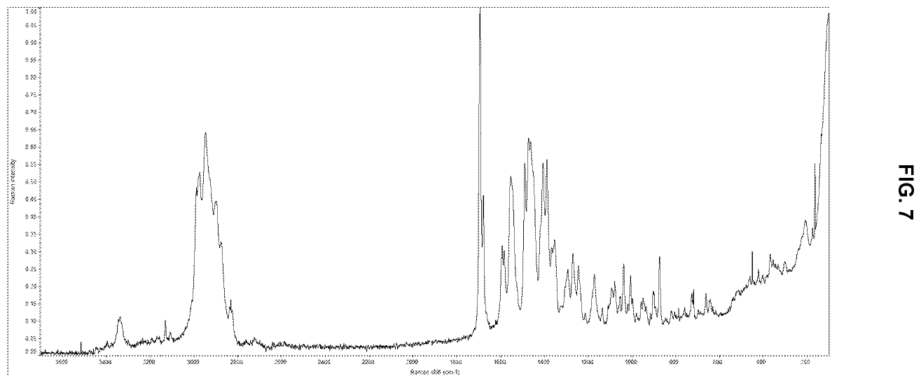

In one embodiment, the invention provides PF-07104091 (Form 2), having a Raman spectrum comprising: (a) one, two, three, four, five, or more than five wavenumber (cm⁻¹) values selected from the group consisting of the values in Table 2 in cm⁻¹±2 cm⁻¹; or (b) wavenumber (cm⁻¹) values essentially the same as in FIG. 7.

In another aspect, the invention provides anhydrous crystalline PF-07104091 (Form 2) characterized by a ¹³C solid state NMR spectrum.

In one embodiment, the invention provides PF-07104091 (Form 2), having a ¹³C solid state NMR spectrum comprising resonance (ppm) values of: 24.1, 39.8 and 41.6 ppm±0.2 ppm.

In one embodiment, the invention provides PF-07104091 (Form 2), having a ¹³C solid state NMR spectrum comprising resonance (ppm) values of: 21.8, 24.1, 39.8 and 41.6 ppm±0.2 ppm.

In one embodiment, the invention provides PF-07104091 (Form 2), having a ¹³C solid state NMR spectrum comprising resonance (ppm) values of: 24.1, 39.8, 41.6 and 138.2 ppm±0.2 ppm.

In another embodiment, the invention provides PF-07104091 (Form 2), having a ¹³C solid state NMR spectrum comprising resonance (ppm) values of: 21.8, 24.1, 39.8, 41.6 and 138.2 ppm±0.2 ppm.

In one embodiment, the invention provides PF-07104091 (Form 2), having a ¹³C solid state NMR spectrum comprising resonance (ppm) values of: 24.1, 39.8 and 41.6 ppm±0.2 ppm; and one or two resonance (ppm) values selected from the group consisting of: 21.8 and 138.2 ppm±0.2 ppm.

In one embodiment, the invention provides PF-07104091 (Form 2), having a ¹³C solid state NMR spectrum comprising resonance (ppm) values of:

24.1, 39.8 and 41.6 ppm±0.2 ppm; and optionally one or two peaks selected from the group consisting of: 21.8 and 138.2 ppm±0.2 ppm.

In another embodiment, the invention provides PF-07104091 (Form 2), having a ¹³C solid state NMR spectrum comprising three or more resonance (ppm) values selected from the group consisting of: 21.8, 24.1, 39.8, 41.6 and 138.2 ppm±0.2 ppm.

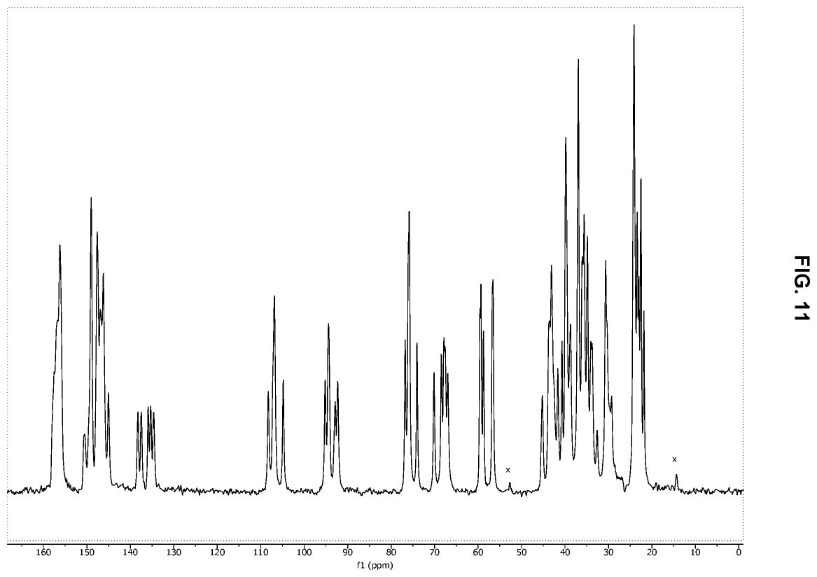

In another embodiment, the invention provides PF-07104091 (Form 2), having a ¹³C solid state NMR spectrum (ppm) comprising: (a) one, two, three, four, five, or more than five resonance (ppm) values selected from the group consisting of the values in Table 3 in ppm±0.2 ppm; or (b) resonance (ppm) values essentially the same as in FIG. 11.

In another aspect, the invention provides anhydrous crystalline PF-07104091 (Form 2), having:
(a) a powder X-ray diffraction (PXRD) pattern comprising peaks at 2θ values of: 9.8, 13.3 and 17.4 °2θ±0.2 °2θ;
(b) a Raman spectrum comprising wavenumber (cm⁻¹) values of: 1691, 1582 and 996 cm⁻¹+2 cm⁻¹, or
(c) a ¹³C solid state NMR spectrum comprising resonance (ppm) values of: 24.1, 39.8 and 41.6 ppm±0.2 ppm; or
or any combination of two or more of (a), (b) and (c).

In another aspect, the invention provides anhydrous crystalline PF-07104091 (Form 2), having:
(a) a powder X-ray diffraction (PXRD) pattern comprising peaks at 2θ values of: 9.8 and 13.3 °2θ±0.2 °2θ; and optionally further comprising a peak at a 2θ value of: 17.4 °2θ±0.2 °2θ;
(b) a Raman spectrum comprising a wavenumber (cm⁻¹) value of: 1691 cm⁻¹±2 cm⁻¹, and optionally further comprising wavenumber (cm⁻¹) values of: 1582 and 996 cm⁻¹±2 cm⁻¹; or
(c) a ¹³C solid state NMR spectrum comprising resonance (ppm) values of: 24.1, 39.8 and 41.6 ppm±0.2 ppm;
or any combination of two or more of (a), (b), and (c).

In another aspect, the invention provides anhydrous crystalline PF-07104091 (Form 2), having:
(1) a powder X-ray diffraction (PXRD) pattern comprising peaks at 2θ values of:
   (a) 9.8, 13.3 and 17.4 °2θ±0.2 °2θ;
   (b) 4.2, 9.8, 13.3 and 17.4 °2θ±0.2 °2θ;
   (c) 7.5, 9.8, 13.3 and 17.4 °2θ±0.2 °2θ; or
   (d) 4.2, 7.5, 9.8, 13.3 and 17.4 °2θ±0.2 °2θ;
(2) a Raman spectrum comprising wavenumber (cm⁻¹) values of:
   (a) 1691, 1582 and 996 cm⁻¹±2 cm⁻¹;
   (b) 1691, 1582, 1036 and 996 cm⁻¹±2 cm⁻¹;
   (c) 1691, 1582, 1365 and 996 cm⁻¹±2 cm⁻¹; or
   (d) 1691, 1582, 1365, 1036 and 996 cm⁻¹±2 cm⁻¹; or
(3) a ¹³C solid state NMR spectrum comprising resonance (ppm) values of:
   (a) 24.1, 39.8 and 41.6 ppm±0.2 ppm;
   (b) 21.8, 24.1, 39.8 and 41.6 ppm±0.2 ppm;
   (c) 24.1, 39.8, 41.6 and 138.2 ppm±0.2 ppm; or
   (d) 21.8, 24.1, 39.8, 41.6 and 138.2 ppm±0.2 ppm.
or any combination of two or more of (1)(a)-(d), (2)(a)-(d) and (3)(a)-(d).

In some embodiments of each of the aspects and embodiments of PF-07104091 (Form 2) herein, the crystalline form is substantially pure anhydrous crystalline PF-07104091 (Form 2).

In another aspect, the invention provides a pharmaceutical composition comprising anhydrous crystalline PF-07104091 (Form 2), according to the aspects or embodiments described herein, and a pharmaceutically acceptable carrier or excipient.

In another aspect, the invention provides a method of treating cancer in a subject in need thereof, comprising administering to the subject a therapeutically effective amount of anhydrous crystalline PF-07104091 (Form 2), or a pharmaceutical composition comprising anhydrous crystalline PF-07104091 (Form 2), according to the aspects or embodiments described herein.

In another aspect, the invention provides a method of treating cancer in a subject in need thereof, comprising administering to the subject an amount of anhydrous crystalline PF-07104091 (Form 2), or a pharmaceutical composition comprising anhydrous crystalline PF-07104091 (Form 2), according to the aspects or embodiments described herein, and an amount of an additional anticancer agent, wherein the amounts of PF-07104091 (Form 2) and the additional anticancer agent together are effective in treating cancer.

In another aspect, the invention provides anhydrous crystalline PF-07104091 (Form 2), or a pharmaceutical composition comprising anhydrous crystalline PF-07104091 (Form 2), according to the aspects or embodiments described herein, for use in the treatment of cancer.

In yet another aspect, the invention provides anhydrous crystalline PF-07104091 (Form 2), according to the aspects or embodiments described herein, for use in the manufacture of a medicament for the treatment of cancer.

In another aspect, the invention provides use of anhydrous crystalline PF-07104091 (Form 2), or a pharmaceutical composition comprising anhydrous crystalline PF-07104091 (Form 2), according to the aspects or embodiments described herein, for the treatment of cancer.

In yet another aspect, the invention provides use of anhydrous crystalline PF-07104091 (Form 2), according to the aspects or embodiments described herein, in the manufacture of a medicament for the treatment of cancer.

In each of the aspects and embodiments of anhydrous crystalline PF-07104091 (Form 2) described herein, the crystalline form may be substantially pure anhydrous crystalline PF-07104091 (Form 2).

Each of the embodiments described herein for anhydrous crystalline PF-07104091 (Form 2) may be combined with other such embodiments, provided the embodiments are not inconsistent with each other.

In a preferred aspect, the invention provides crystalline PF-07104091 monohydrate (Form 3). In some embodiments, crystalline PF-07104091 monohydrate (Form 3) is characterized by its powder X-ray diffraction (PXRD) pattern. In other embodiments, crystalline PF-07104091 monohydrate (Form 3) is characterized by its Raman spectrum. In other embodiments, crystalline PF-07104091 monohydrate (Form 3) is characterized by its ¹³C solid state NMR spectrum.

In further embodiments, crystalline PF-07104091 monohydrate (Form 3) is characterized by any combination of two or more of these methods. Exemplary combinations including two or more of the following are provided herein: powder X-ray diffraction (PXRD) pattern (2θ); Raman spectrum wavenumber values (cm⁻¹); or ¹³C solid state NMR spectrum (ppm). In some embodiments crystalline PF-07104091 monohydrate (Form 3) is characterized by PXRD and Raman. In other embodiments, crystalline PF-07104091 monohydrate (Form 3) is characterized by PXRD and ¹³C solid state NMR. In other embodiments crystalline PF-07104091 monohydrate (Form 3) is characterized by Raman and ¹³C solid state NMR. In other embodiments crystalline PF-07104091 monohydrate (Form 3) is characterized by PXRD, Raman and ¹³C solid state NMR.

In one aspect, the invention provides crystalline PF-07104091 monohydrate (Form 3) characterized by a powder X-ray diffraction (PXRD) pattern.

In one embodiment, the invention provides crystalline PF-07104091 monohydrate (Form 3) having a powder X-ray diffraction (PXRD) pattern comprising peaks at 2θ values of: 8.4, 10.1 and 21.5 °2θ±0.2 °2θ.

In one embodiment, the invention provides crystalline PF-07104091 monohydrate (Form 3) having a powder X-ray diffraction (PXRD) pattern comprising peaks at 2θ values of: 8.4, 10.1, 16.9 and 21.5 °2θ±0.2 °2θ.

In one embodiment, the invention provides crystalline PF-07104091 monohydrate (Form 3) having a powder X-ray diffraction (PXRD) pattern comprising peaks at 2θ values of: 8.4, 10.1, 21.5 and 27.0 °2θ±0.2 °2θ0.

In another embodiment, the invention provides crystalline PF-07104091 monohydrate (Form 3) having a powder X-ray diffraction (PXRD) pattern comprising peaks at 2θ values of: 8.4, 10.1, 16.9, 21.5 and 27.0 °2θ±0.2 °2θ.

In another embodiment, the invention provides crystalline PF-07104091 monohydrate (Form 3) having a powder X-ray diffraction (PXRD) pattern comprising peaks at 2θ values of:
(a) 8.4, 10.1 and 21.5 °2θ±0.2 °2θ;
(b) 8.4, 10.1, 16.9 and 21.5 °2θ±0.2 °2θ,
(c) 8.4, 10.1, 21.5 and 27.0 °2θ±0.2 °2θ; or
(d) 8.4, 10.1, 16.9, 21.5 and 27.0 °2θ±0.2 °2θ.

In one embodiment, the invention provides crystalline PF-07104091 monohydrate (Form 3) having a powder X-ray diffraction (PXRD) pattern comprising peaks at 2θ values of: 8.4, 10.1 and 21.5 °2θ±0.2 °2θ; and optionally one or two peaks selected from the group consisting of: 16.9 and 27.0 °2θ±0.2 °2θ.

In another embodiment, the invention provides crystalline PF-07104091 monohydrate (Form 3) having a PXRD pattern comprising three or more peaks at 2θ values selected from the group consisting of 8.4, 10.1, 16.9, 21.5 and 27.0 °2θ±0.2 °2θ.

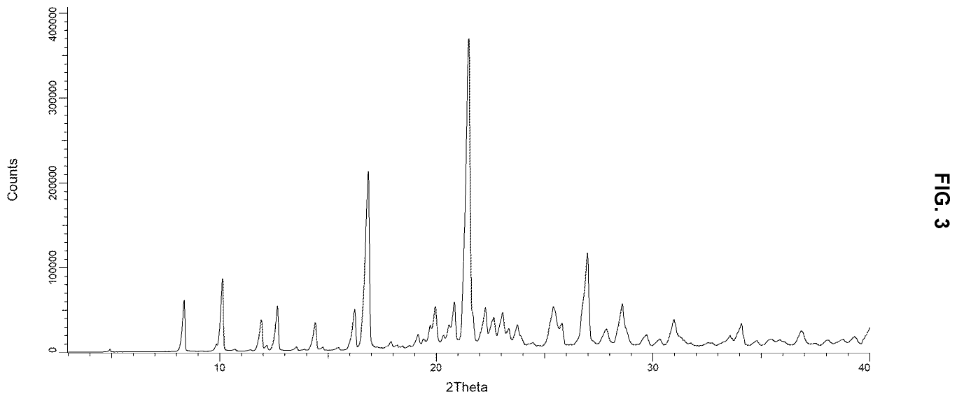

In another embodiment, the invention provides crystalline PF-07104091 monohydrate (Form 3) having a PXRD pattern comprising: (a) one, two, three, four, five, or more than five peaks selected from the group consisting of the peaks in Table 4 in °2θ±0.2 °2θ; or (b) peaks at 2θ values essentially the same as in FIG. 3.

In another aspect, the invention provides crystalline PF-07104091 monohydrate (Form 3) characterized by a Raman spectrum.

In one embodiment, the invention provides crystalline PF-07104091 monohydrate (Form 3) having a Raman spectrum comprising wavenumber (cm⁻¹) values of: 1657, 1595 and 1408 cm⁻¹+2 cm⁻¹.

In another embodiment, the invention provides crystalline PF-07104091 monohydrate (Form 3) having a Raman spectrum comprising wavenumber (cm⁻¹) values of: 1657, 1595, 1408 and 923 cm⁻¹±2 cm⁻¹.

In another embodiment, the invention provides crystalline PF-07104091 monohydrate (Form 3) having a Raman spectrum comprising wavenumber (cm⁻¹) values of: 1657, 1595, 1408 and 1272 cm⁻¹±2 cm⁻¹.

In another embodiment, the invention provides crystalline PF-07104091 monohydrate (Form 3) having a Raman spectrum comprising wavenumber (cm⁻¹) values of: 1657, 1595, 1408, 1272 and 923 cm⁻¹±2 cm⁻¹.

In another embodiment, the invention provides crystalline PF-07104091 monohydrate (Form 3) having a Raman spectrum comprising wavenumber (cm⁻¹) values of:
(a) 1657, 1595 and 1408 cm⁻¹±2 cm⁻¹;
(b) 1657, 1595, 1408 and 923 cm⁻¹±2 cm⁻¹;
(c) 1657, 1595, 1408 and 1272 cm⁻¹±2 cm⁻¹, or
(d) 1657, 1595, 1408, 1272 and 923 cm⁻¹±2 cm⁻¹.

In one embodiment, the invention provides crystalline PF-07104091 monohydrate (Form 3) having a Raman spectrum comprising wavenumber (cm⁻¹) values of: 1657, 1595 and 1408 cm⁻¹±2 cm⁻¹, and one or two peaks selected from the group consisting of: 1272 and 923 cm⁻¹+2 cm⁻¹

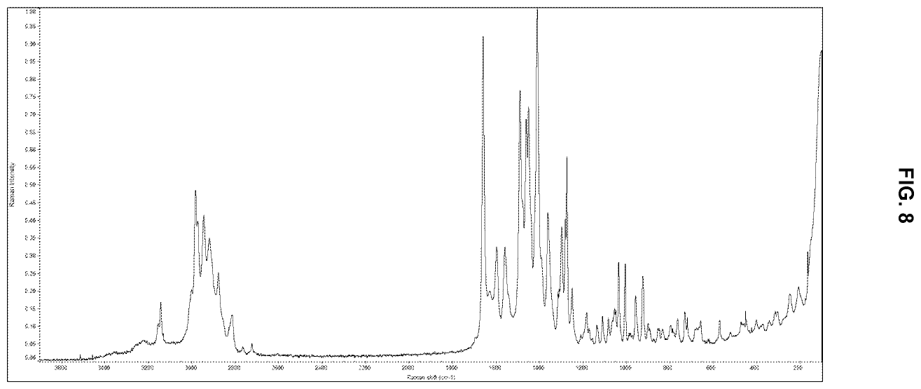

In one embodiment, the invention provides crystalline PF-07104091 monohydrate (Form 3) having a Raman spectrum comprising: (a) one, two, three, four, five, or more than five wavenumber (cm⁻¹) values selected from the group consisting of the values in Table 5 in cm⁻¹±2 cm⁻¹; or (b) wavenumber (cm⁻¹) values essentially the same as in FIG. 8.

In another aspect, the invention provides crystalline PF-07104091 monohydrate (Form 3) characterized by a ¹³C solid state NMR spectrum.

In one embodiment, the invention provides crystalline PF-07104091 monohydrate (Form 3) having a ¹³C solid state NMR spectrum comprising resonance (ppm) values of: 25.2 and 37.5 ppm±0.2 ppm.

In one embodiment, the invention provides crystalline PF-07104091 monohydrate (Form 3) having a ¹³C solid state NMR spectrum comprising resonance (ppm) values of: 25.2, 37.5 and 159.3 ppm±0.2 ppm.

In one embodiment, the invention provides crystalline PF-07104091 monohydrate (Form 3) having a ¹³C solid state NMR spectrum comprising resonance (ppm) values of: 25.2, 37.5, 151.9 and 159.3 ppm±0.2 ppm.

In one embodiment, the invention provides crystalline PF-07104091 monohydrate (Form 3) having a ¹³C solid state NMR spectrum comprising resonance (ppm) values of: 25.2, 37.5, 152.5 and 159.3 ppm±0.2 ppm.

In another embodiment, the invention provides crystalline PF-07104091 monohydrate (Form 3) having a ¹³C solid state NMR spectrum comprising resonance (ppm) values of: 25.2, 37.5, 151.9, 152.5 and 159.3 ppm±0.2 ppm.

In one embodiment, the invention provides crystalline PF-07104091 monohydrate (Form 3) having a ¹³C solid state NMR spectrum comprising resonance (ppm) values of: 25.2, 37.5 and 159.3 ppm±0.2 ppm; and one or two resonance (ppm) values selected from the group consisting of: 151.9 and 152.5 ppm±0.2 ppm.

In another embodiment, the invention provides crystalline PF-07104091 monohydrate (Form 3) having a ¹³C solid state NMR spectrum comprising resonance (ppm) values of:
(a) 25.2 and 37.5 ppm±0.2 ppm;
(b) 25.2, 37.5 and 159.3 ppm±0.2 ppm;
(c) 25.2, 37.5, 151.9 and 159.3 ppm±0.2 ppm;
(d) 25.2, 37.5, 152.5 and 159.3 ppm±0.2 ppm; or
(e) 25.2, 37.5, 151.9, 152.5 and 159.3 ppm±0.2 ppm.

In one embodiment, the invention provides crystalline PF-07104091 monohydrate (Form 3) having a ¹³C solid state NMR spectrum comprising resonance (ppm) values of: 25.2, 37.5 and 159.3 ppm±0.2 ppm; and optionally one or two peaks selected from the group consisting of: 151.9 and 152.5 ppm±0.2 ppm.

In another embodiment, the invention provides crystalline PF-07104091 monohydrate (Form 3) having a ¹³C solid state NMR spectrum comprising three or more resonance (ppm) values selected from the group consisting of: 25.2, 37.5, 151.9, 152.5 and 159.3 ppm±0.2 ppm.

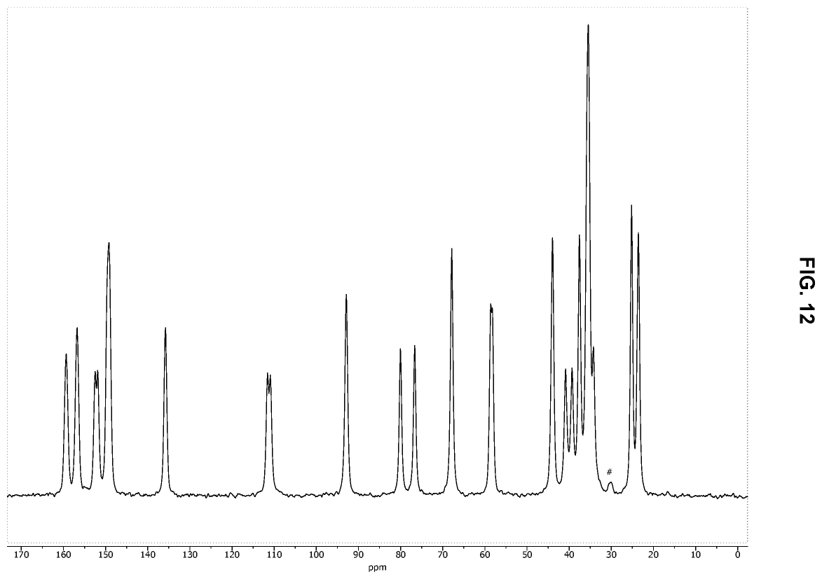

In another embodiment, the invention provides crystalline PF-07104091 monohydrate (Form 3) having a ¹³C solid state NMR spectrum (ppm) comprising: (a) one, two, three, four, five, or more than five resonance (ppm) values selected from the group consisting of the values in Table 6 in ppm±0.2 ppm; or (b) resonance (ppm) values essentially the same as in FIG. 12.

In another aspect, the invention provides crystalline PF-07104091 monohydrate (Form 3) having:
(a) a powder X-ray diffraction (PXRD) pattern comprising peaks at 2θ values of: 8.4, 10.1 and 21.5 °2θ±0.2 °2θ;
(b) a Raman spectrum comprising wavenumber (cm⁻¹) values of: 1657, 1595 and 1408 cm⁻¹+2 cm⁻¹; or
(c) a ¹³C solid state NMR spectrum comprising resonance (ppm) values of: 25.2, 37.5 and 159.3 ppm±0.2 ppm; or
or any combination of two or more of (a), (b) and (c).

In another aspect, the invention provides crystalline PF-07104091 monohydrate (Form 3), having:
(a) a powder X-ray diffraction (PXRD) pattern comprising peaks at 2θ values of: 8.4 and 10.1 °2θ±0.2 °2θ; and optionally further comprising a peak at a 2θ value of: 21.5 °2θ±0.2 °2θ;
(b) a Raman spectrum comprising a wavenumber (cm⁻¹) value of: 1657 cm⁻¹±2 cm⁻¹; and optionally further comprising wavenumber (cm⁻¹) values of: 1595 and 1408 cm⁻¹±2 cm⁻¹, or
(c) a ¹³C solid state NMR spectrum comprising resonance (ppm) values of: 25.2 and 37.5 ppm±0.2 ppm; and optionally further comprising a resonance (ppm) value of: 159.3 ppm±0.2 ppm
or any combination of two or more of (a), (b), and (c).

In another aspect, the invention provides crystalline PF-07104091 monohydrate (Form 3) having:
(1) a powder X-ray diffraction (PXRD) pattern comprising peaks at 2θ values of:
   (a) 8.4, 10.1 and 21.5 °2θ±0.2 °2θ;
   (b) 8.4, 10.1, 16.9 and 21.5 °2θ±0.2 °2θ;
   (c) 8.4, 10.1, 21.5 and 27.0 °2θ±0.2 °2θ; or
   (d) 8.4, 10.1, 16.9, 21.5 and 27.0 °2θ±0.2 °2θ;
(2) a Raman spectrum comprising wavenumber (cm⁻¹) values of:
   (a) 1657, 1595 and 1408 cm⁻¹±2 cm⁻¹;
   (b) 1657, 1595, 1408 and 923 cm⁻¹±2 cm⁻¹;
   (c) 1657, 1595, 1408 and 1272 cm⁻¹±2 cm⁻¹, or
   (d) 1657, 1595, 1408, 1272 and 923 cm⁻¹±2 cm⁻¹; or
(3) a ¹³C solid state NMR spectrum comprising resonance (ppm) values of:
   (a) 25.2 and 37.5 ppm±0.2 ppm;
   (b) 25.2, 37.5 and 159.3 ppm±0.2 ppm;
   (c) 25.2, 37.5, 151.9 and 159.3 ppm±0.2 ppm;
   (d) 25.2, 37.5, 152.5 and 159.3 ppm±0.2 ppm; or
   (e) 25.2, 37.5, 151.9, 152.5 and 159.3 ppm±0.2 ppm;
or any combination of two or more of (1)(a)-(d), (2)(a)-(d), and (3)(a)-(e).

In another aspect, the invention provides a pharmaceutical composition comprising crystalline PF-07104091 monohydrate (Form 3), according to the aspects or embodiments described herein, and a pharmaceutically acceptable carrier or excipient.

In another aspect, the invention provides a method of treating cancer in a subject in need thereof, comprising administering to the subject a therapeutically effective amount of crystalline PF-07104091 monohydrate (Form 3), or a pharmaceutical composition comprising crystalline PF-07104091 monohydrate (Form 3), according to the aspects or embodiments described herein.

In another aspect, the invention provides a method of treating cancer in a subject in need thereof, comprising administering to the subject an amount of crystalline PF-07104091 monohydrate (Form 3), or a pharmaceutical composition comprising crystalline PF-07104091 monohydrate (Form 3), according to the aspects or embodiments described herein, and an amount of an additional anticancer agent, wherein the amounts of PF-07104091 monohydrate (Form 3) and the additional anticancer agent together are effective in treating cancer.

In another aspect, the invention provides crystalline PF-07104091 monohydrate (Form 3), or a pharmaceutical composition comprising crystalline PF-07104091 monohydrate (Form 3), according to the aspects or embodiments described herein, for use in the treatment of cancer.

In yet another aspect, the invention provides crystalline PF-07104091 monohydrate (Form 3), according to the aspects or embodiments described herein, for use in the manufacture of a medicament for the treatment of cancer.

In another aspect, the invention provides use of crystalline PF-07104091 monohydrate (Form 3), or a pharmaceutical composition comprising crystalline PF-07104091 monohydrate (Form 3), according to the aspects or embodiments described herein, for the treatment of cancer.

In yet another aspect, the invention provides use of crystalline PF-07104091 monohydrate (Form 3), according to the aspects or embodiments described herein, in the manufacture of a medicament for the treatment of cancer.

In each of the aspects and embodiments of crystalline PF-07104091 monohydrate (Form 3) described herein, the crystalline form may be substantially pure crystalline PF-07104091 monohydrate (Form 3).

Each of the embodiments described herein for crystalline PF-07104091 monohydrate (Form 3) may be combined with other such embodiments, provided the embodiments are not inconsistent with each other.

In another aspect, the invention provides amorphous PF-07104091 (Form 4).

In some embodiments, the invention provides amorphous PF-07104091 (Form 4), having a powder X-ray diffraction (PXRD) pattern comprising a broad peak at diffraction angles (2θ) from about 5 to about 35 °2θ±0.2 °2θ.

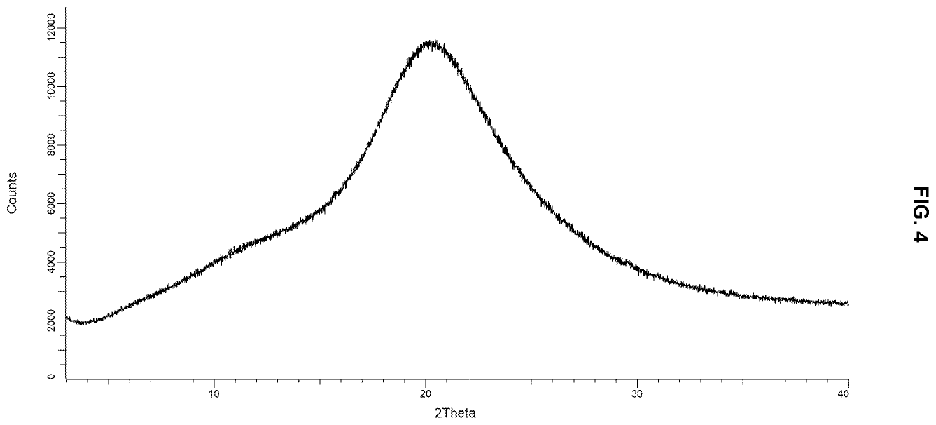

In some embodiments, the invention provides amorphous PF-07104091 (Form 4), having a powder X-ray diffraction (PXRD) pattern essentially the same as in FIG. 4.

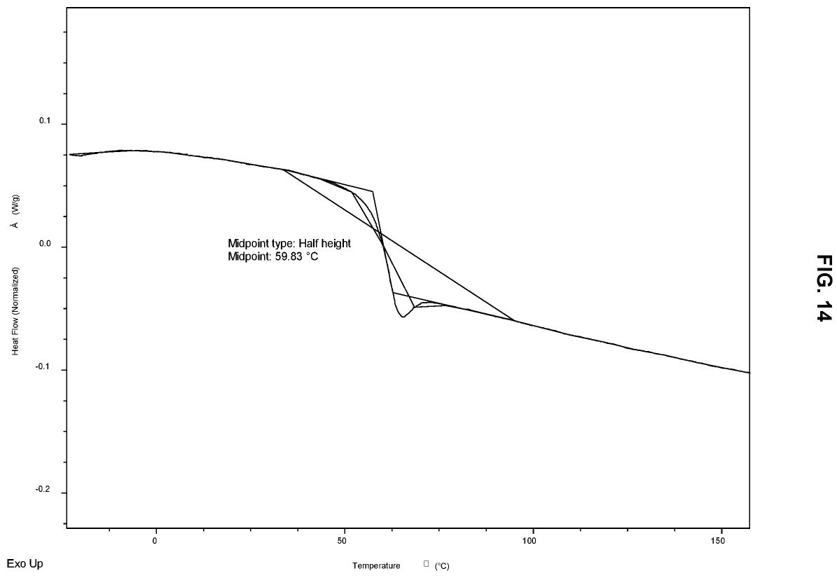

In some embodiments, the invention provides amorphous PF-07104091 (Form 4), having a glass transition temperature (T_{g}) of 59.8±5° C. (FIG. 14).

In another embodiment, the invention provides amorphous PF-07104091 (Form 4), having:
(1) a powder X-ray diffraction (PXRD) pattern (2θ) comprising:
   (a) a broad peak at diffraction angles (2θ) from about 5 to about 35 °2θ±0.2 °2θ; or
   (b) peaks at 2θ values essentially the same as in FIG. 4; or
(2) a DSC thermogram comprising:
   (a) a glass transition temperature (T_{g}) of about 59.8±5° C. (as measured by DSC at a ramp rate of 10° C./min); or
   (b) a DSC thermogram essentially the same as in FIG. 14;
   or any combination of two or more of (1)(a)-(b) and (2)(a)-(b).

In another aspect, the invention provides a pharmaceutical composition comprising amorphous PF-07104091 (Form 4), according to the aspects or embodiments described herein, and a pharmaceutically acceptable carrier or excipient.

In another aspect, the invention provides a method of treating cancer in a subject in need thereof, comprising administering to the subject a therapeutically effective amount of amorphous PF-07104091 (Form 4), or a pharmaceutical composition comprising amorphous PF-07104091 (Form 4), according to the aspects or embodiments described herein.

In another aspect, the invention provides a method of treating cancer in a subject in need thereof, comprising administering to the subject an amount of amorphous PF-07104091 (Form 4), or a pharmaceutical composition comprising amorphous PF-07104091 (Form 4), according to the aspects or embodiments described herein, and an amount of an additional anticancer agent, wherein the amounts of amorphous PF-07104091 (Form 4) and the additional anticancer agent together are effective in treating cancer.

In another aspect, the invention provides amorphous PF-07104091 (Form 4), or a pharmaceutical composition comprising amorphous PF-07104091 (Form 4), according to the aspects or embodiments described herein, for use in the treatment of cancer.

In yet another aspect, the invention provides amorphous PF-07104091 (Form 4), according to the aspects or embodiments described herein, for use in the manufacture of a medicament for the treatment of cancer.

In another aspect, the invention provides use of amorphous PF-07104091 (Form 4), or a pharmaceutical composition comprising amorphous PF-07104091 (Form 4), according to the aspects or embodiments described herein, for the treatment of cancer.

In yet another aspect, the invention provides use of amorphous PF-07104091 (Form 4), according to the aspects or embodiments described herein, in the manufacture of a medicament for the treatment of cancer.

In each of the aspects and embodiments of amorphous PF-07104091 (Form 4) described herein, the amorphous form may be substantially pure amorphous PF-07104091 (Form 4).

Each of the embodiments described herein for amorphous PF-07104091 (Form 4) may be combined with other such embodiments, provided the embodiments are not inconsistent with each other.

In a further aspect, the invention provides anhydrous crystalline PF-07104091 (Form 5). Form 5 was prepared by dehydration of PF-07104091 monohydrate (Form 3).

In some embodiments, PF-07104091 (Form 5) is characterized by its powder X-ray diffraction (PXRD) pattern. In other embodiments, PF-07104091 (Form 5) is characterized by its Raman spectrum. In other embodiments, PF-07104091 (Form 5) is characterized by its ¹³C solid state NMR spectrum.

In further embodiments, PF-07104091 (Form 5) is characterized by any combination of two or more of these methods. Exemplary combinations including two or more of the following are provided herein: powder X-ray diffraction (PXRD) pattern (2θ); Raman spectrum wavenumber values (cm⁻¹); or ¹³C solid state NMR spectrum (ppm). In some embodiments, PF-07104091 (Form 5) is characterized by PXRD and Raman. In other embodiments, PF-07104091 (Form 5) is characterized by PXRD and ¹³C solid state NMR. In other embodiments, PF-07104091 (Form 5) is characterized by Raman and ¹³C solid state NMR. In other embodiments, crystalline PF-07104091 (Form 5) is characterized by PXRD, Raman and ¹³C solid state NMR.

In one aspect, the invention provides anhydrous crystalline PF-07104091 (Form 5) characterized by a powder X-ray diffraction (PXRD) pattern.

In another embodiment, the invention provides anhydrous crystalline PF-07104091 (Form 5), having a PXRD pattern comprising three or more peaks at 2θ values selected from the group consisting of: 10.2, 12.4, 15.4, 17.2, 17.9, 19.8, 21.6, 22.5, 23.7 and 26.2 °2θ±0.2 °2θ.

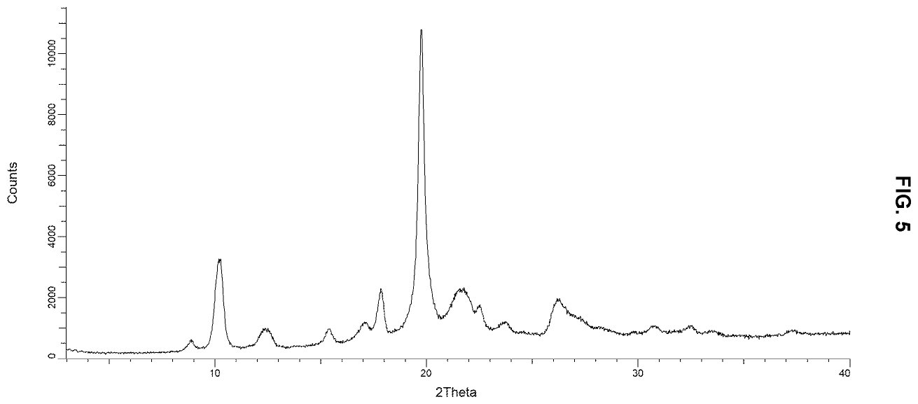

In another embodiment, the invention provides anhydrous crystalline PF-07104091 (Form 5), having a PXRD pattern comprising: (a) one, two, three, four, five, or more than five peaks selected from the group consisting of the peaks in Table 8 in °2θ±0.2 °2θ; or (b) peaks at 2θ values essentially the same as in FIG. 5.

In another aspect, the invention provides anhydrous crystalline PF-07104091 (Form 5), characterized by a Raman spectrum.

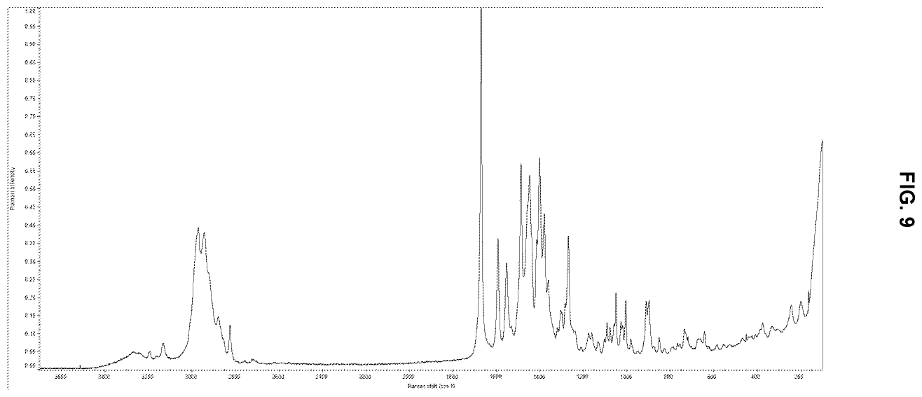

In one embodiment, the invention provides anhydrous crystalline PF-07104091 (Form 5), having a Raman spectrum comprising: (a) one, two, three, four, five, or more than five wavenumber (cm⁻¹) values selected from the group consisting of the values in Table 9 in cm⁻¹1±2 cm⁻¹; or (b) wavenumber (cm⁻¹) values essentially the same as in FIG. 9.

In another aspect, the invention provides anhydrous crystalline PF-07104091 (Form 5), characterized by a ¹³C solid state NMR spectrum.

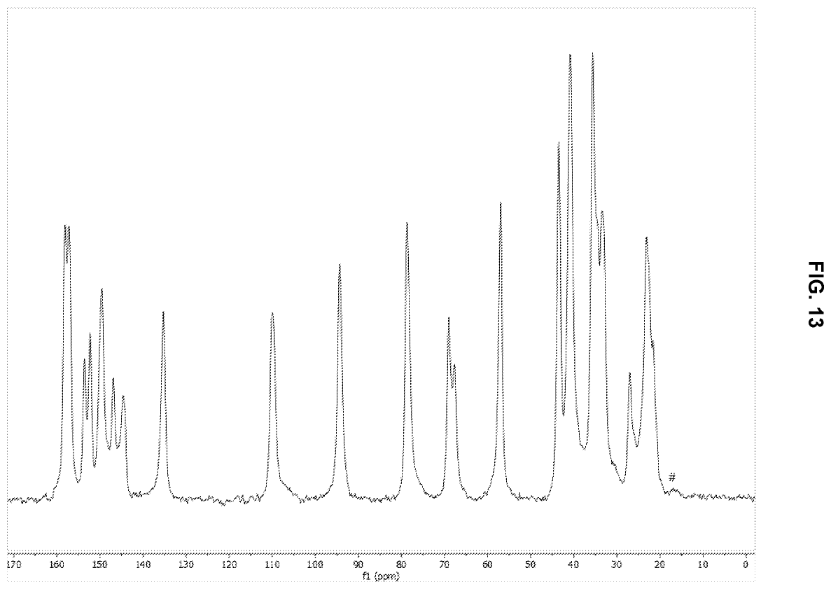

In some such embodiments, the invention provides anhydrous crystalline PF-07104091 (Form 5), having a ¹³C solid state NMR spectrum (ppm) comprising: (a) one, two, three, four, five, or more than five resonance (ppm) values selected from the group consisting of the values in Table 10 in ppm±0.2 ppm; or (b) resonance (ppm) values essentially the same as in FIG. 13.

In another aspect, the invention provides anhydrous crystalline PF-07104091 (Form 5), having:
(a) peaks at 2θ values essentially the same as in FIG. 5;
(b) wavenumber (cm⁻¹) values essentially the same as in FIG. 9; or
(c) resonance (ppm) values essentially the same as in FIG. 13; or
or any combination of two or more of (a), (b) and (c).

In another aspect, the invention provides a pharmaceutical composition comprising anhydrous crystalline PF-07104091 (Form 5), according to the aspects or embodiments described herein, and a pharmaceutically acceptable carrier or excipient.

In another aspect, the invention provides a method of treating cancer in a subject in need thereof, comprising administering to the subject a therapeutically effective amount of anhydrous crystalline PF-07104091 (Form 5), or a pharmaceutical composition comprising anhydrous crystalline PF-07104091 (Form 5), according to the aspects or embodiments described herein.

In another aspect, the invention provides a method of treating cancer in a subject in need thereof, comprising administering to the subject an amount of anhydrous crystalline PF-07104091 (Form 5), or a pharmaceutical composition comprising anhydrous crystalline PF-07104091 (Form 5), according to the aspects or embodiments described herein, and an amount of an additional anticancer agent, wherein the amounts of PF-07104091 (Form 5) and the additional anticancer agent together are effective in treating cancer.

In another aspect, the invention provides anhydrous crystalline PF-07104091 (Form 5), or a pharmaceutical composition comprising anhydrous crystalline PF-07104091 (Form 5), according to the aspects or embodiments described herein, for use in the treatment of cancer.

In yet another aspect, the invention provides anhydrous crystalline PF-07104091 (Form 5), according to the aspects or embodiments described herein, for use in the manufacture of a medicament for the treatment of cancer.

In another aspect, the invention provides use of anhydrous crystalline PF-07104091 (Form 5), or a pharmaceutical composition comprising anhydrous crystalline PF-07104091 (Form 5), according to the aspects or embodiments described herein, for the treatment of cancer.

In yet another aspect, the invention provides use of anhydrous crystalline PF-07104091 (Form 5), according to the aspects or embodiments described herein, in the manufacture of a medicament for the treatment of cancer.

In each of the aspects and embodiments anhydrous crystalline PF-07104091 (Form 5) described herein, the crystalline form may be a substantially pure crystalline form of PF-07104091 (Form 5).

Each of the embodiments described herein for anhydrous crystalline PF-07104091 (Form 5) may be combined with other such embodiments, provided the embodiments are not inconsistent with each other.

In some embodiments of the methods and uses described herein, the cancer is selected from the group consisting of breast cancer, prostate cancer, lung cancer (including non-small cell lung cancer, NSCLC, and small cell lung cancer, SCLC), liver cancer (including hepatocellular carcinoma, HCC), kidney cancer (including renal cell carcinoma, RCC), bladder cancer (including urothelial carcinomas, such as upper urinary tract urothelial carcinoma, UUTUC), ovarian cancer (including epithelial ovarian cancer, EOC), peritoneal cancer (including primary peritoneal cancer, PPC), fallopian tube cancer, cervical cancer, uterine cancer (including endometrial cancer), pancreatic cancer, stomach cancer, colorectal cancer, esophageal cancer, head and neck cancer (including squamous cell carcinoma of the head and neck (SCCHN), thyroid cancer, and salivary gland cancer), testicular cancer, adrenal cancer, skin cancer (including basal cell carcinoma and melanoma), brain cancer (including astrocytoma, meningioma, and glioblastoma), sarcoma (including osteosarcoma and liposarcoma), and lymphoma (including mantle cell lymphoma, MCL).

In some embodiments of the methods and uses described herein, the cancer is SCLC. In some such embodiments, the SCLC is Rb-negative or Rb-deficient.

In some embodiments of the methods and uses described herein, the cancer is NSCLC. In some such embodiments, the NSCLC is characterized by amplification or overexpression of cyclin E1 (CCNE1) and/or cyclin E2 (CCNE2).

In some embodiments of the methods and uses described herein, the cancer is ovarian cancer (including epithelial ovarian cancer, EOC), peritoneal cancer (including primary peritoneal cancer, PPC), or fallopian tube cancer. In some such embodiments, the cancer is characterized by amplification or overexpression of CCNE1 and/or CCNE2.

In some embodiments of the methods and uses described herein, the cancer is TNBC. In some such embodiments, the TNBC is refractory to CDK4/6 inhibitors, such as palbociclib.

In some embodiments of the methods and uses described herein, the cancer is HR-positive, HER2-negative breast cancer, including advanced or metastatic breast cancer. In some such embodiments, the breast cancer is refractory to CDK4/6 inhibitors, such as palbociclib.

In some embodiments of the methods and uses described herein, the cancer is advanced or metastatic cancer. In some embodiments of the methods and uses described herein, the cancer is early stage or non-metastatic cancer.

In other embodiments, the cancer is breast cancer, including, e.g., ER-positive/HR-positive, HER2-negative breast cancer; ER-positive/HR-positive, HER2-positive breast cancer; triple negative breast cancer (TNBC); or inflammatory breast cancer. In some embodiments, the breast cancer demonstrates primary or acquired resistance to endocrine therapy, anti-HER2 targeted agents, CDK4/CDK6 inhibition, or chemotherapy (e.g., taxanes or platins).

In some embodiments, the breast cancer is advanced or metastatic breast cancer.

In some embodiments of each of the foregoing, the breast cancer is characterized by amplification or overexpression of CCNE1 and/or CCNE2.

In some embodiments of the methods provided herein, the abnormal cell growth is cancer characterized by amplification or overexpression of CCNE1 and/or CCNE2. In some embodiments of the methods provided herein, the subject is identified as having a cancer characterized by amplification or overexpression of CCNE1 and/or CCNE2.

In some embodiments, the cancer is breast cancer or ovarian cancer. In some such embodiments, the cancer is breast cancer or ovarian cancer characterized by amplification or overexpression of CCNE1 and/or CCNE2. In some such embodiments, the cancer is (a) breast cancer or ovarian cancer; (b) characterized by amplification or overexpression of CCNE1 or CCNE2; or (c) both (a) and (b).

In some embodiments, the compound of the invention is administered as first line therapy. In other embodiments, the compound of the invention is administered as second (or later) line therapy.

In some embodiments, the compound of the invention is administered as second (or later) line therapy following treatment with an endocrine therapy and/or a CDK4/6 inhibitor. In some embodiments, the compound of the invention is administered as second (or later) line therapy following treatment with an endocrine therapy, e.g., an aromatase inhibitor, a SERM or a SERD. In some embodiments, the compound of the invention is administered as second (or later) line therapy following treatment with a CDK4/6 inhibitor (e.g., palbociclib, ribociclib or abemaciclib, or a pharmaceutically acceptable salt thereof). In some embodiments, the compound of the invention is administered as second (or later) line therapy following treatment with one or more chemotherapy regimens (e.g., including taxanes or platinum agents). In some embodiments, the compound of the invention is administered as second (or later) line therapy following treatment with anti-HER2 targeted agents (e.g., trastuzumab, pertuzumab, lapatinib, or ado-trastuzumab emtansine (T-DM1)).

As used herein, an “effective dosage”, “effective amount” or “therapeutically effective amount” of a compound or pharmaceutical composition is the amount that, when used as indicated (which may be alone if used as a single agent or together with other agents if used in combination) is sufficient to affect one or more beneficial or desired outcomes, including preventing, ameliorating or treating the biochemical, histological or behavioral symptoms of the disease, its complications, and intermediate pathological phenotypes presenting during development of the disease. For prophylactic use, beneficial or desired outcomes may include: eliminating or reducing the risk, lessening the severity, or delaying the onset of the disease. For therapeutic use, beneficial or desired outcomes may include: reducing the incidence or ameliorating one or more symptoms of the disease, reducing the dose of another medication used to treat the disease, enhancing the efficacy or safety of another medication used to treat the disease, or delaying the time to disease progression.

In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or (7) delaying the progression of the disease in a patient.

An effective dosage can be administered in one or more administrations. For the purposes of this invention, an effective dosage of a drug, compound, or pharmaceutical composition is an amount sufficient to accomplish prophylactic or therapeutic treatment either directly or indirectly. As is understood in the clinical context, an effective dosage of a drug, compound or pharmaceutical composition may or may not be achieved in conjunction with another drug, compound or pharmaceutical composition.

A “non-standard dosing regimen” refers to a regimen for administering an amount of a substance, agent, compound or pharmaceutical composition, which is different from the amount, dose or schedule typically used for that substance, agent, compound or pharmaceutical composition in a clinical or therapeutic setting. A “non-standard dosing regimen”, includes a “non-standard dose” or a “non-standard dosing schedule.”

A “low dose amount regimen” refers to a dosing regimen where the amount of one or more of the substances, agents, compounds or pharmaceutical compositions in the regimen is dosed at a lower amount or dose than typically used in a clinical or therapeutic setting for that agent, for example when that agent is dosed as a single agent therapy.

The retinoblastoma susceptibility gene (RB1) was the first tumor suppressor gene to be molecularly defined. The retinoblastoma gene product, RB, is frequently mutated or deleted in retinoblastoma and osteosarcoma, and is mutated or deleted with variable frequency in other tumor types, such as prostate cancer (including neuroendocrine prostate carcinoma), breast cancer (including triple negative breast cancer, TNBC), lung cancer (including small cell lung cancer, SCLC, and non-small cell lung cancer, NSCLC), liver cancer, bladder cancer, ovarian cancer, uterine cancer, cervical cancer, stomach cancer, esophageal cancer, head and neck cancer, glioblastoma, and lymphoma. In human cancers, the function of RB may be disrupted through neutralization by a binding protein, (e.g., the human papilloma virus-E7 protein in cervical carcinoma; Ishiji, T, 2000[0021], J Dermatol., 27: 73-86) or deregulation of pathways ultimately responsible for its phosphorylation.

By “RB pathway” it is meant the entire pathway of molecular signaling that includes retinoblastoma protein (RB), and other protein/protein families in the pathway, including but not limited to CDK, E2f, atypical protein kinase C, and Skp2. Inactivation of the RB pathway often results from perturbation of p16INK4a, Cyclin D1, and CDK4.

The terms “RB+,” “RB plus,” “RB-proficient” or “RB-positive” may be used to describe cells expressing detectable amounts of functional RB protein. RB-positive includes wild-type and non-mutated RB protein. A wild-type RB (RB-WT) is generally understood to mean that form of the RB protein which is normally present in a corresponding population and which has the function which is currently assigned to this protein. RB-positive may be cells which contain a functional RB gene. Cells which are RB-positive may also be cells that can encode a detectable RB protein function.

The terms “RB-,” “RB minus,” “RB-deficient” or “RB-negative” describe several types of cell where the function of RB is disrupted, including cells which produce no detectable amounts of functional RB protein. Cells that are RB-negative may be cells which do not contain a functional RB gene. Cells that are RB-negative may also be cells that can encode an RB protein, but in which the protein does not function properly.

In some embodiments of each of the methods and uses described herein, the cancer is characterized as retinoblastoma wild type (RB-WT). In some embodiments of each of the methods and uses described herein, the cancer is characterized as RB-positive or RB-proficient. Such RB-positive or RB-proficient cancers contain at least some functional retinoblastoma genes. In some embodiments, such RB-WT, RB-positive or RB-proficient cancers are characterized as RB1-WT, RB1 -positive or RB1-proficient cancers.

In some embodiments of each of the methods and uses described herein, the cancer is characterized as RB-negative or RB-deficient. Such RB-negative or RB-deficient cancers may be characterized by loss of function mutations, which may encode missense mutations (i.e., encode the wrong amino acid) or nonsense mutations (i.e., encode a stop codon). Alternatively, such RB-negative cancers may be characterized by deletion of all or part of the retinoblastoma gene. In some embodiments, such RB-negative or RB-deficient cancers are characterized as RB1-negative or RB1-deficient.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

“Tumor burden” or “tumor load’, refers to the total amount of tumorous material distributed throughout the body. Tumor burden refers to the total number of cancer cells or the total size of tumor(s), throughout the body, including lymph nodes and bone marrow. Tumor burden can be determined by a variety of methods known in the art, such as, e.g., using calipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CR or MRI scans.

The term “patient” or “subject” refer to any single subject for which therapy is desired or that is participating in a clinical trial, epidemiological study or used as a control, including humans and mammalian veterinary patients such as cattle, horses, dogs and cats. In some embodiments, the subject is a human.

In some embodiments of each of the methods and uses described herein, the patient or subject is an adult human. In some embodiments, the subject is a woman of any menopausal status or a man. In some embodiments, the subject is a post-menopausal woman or a man. In some embodiments, the subject is a post-menopausal woman. In some embodiments, the subject is a pre-menopausal or peri-menopausal woman. In some embodiments, the subject is a pre-menopausal or peri-menopausal woman treated with a luteinizing hormone-releasing hormone (LHRH) agonist. In some such embodiments, the subject is a man. In some embodiments, the subject is a man treated with an LHRH or gonadotropin-releasing hormone (GnRH) agonist.

The terms “treat” or “treating” of a cancer as used herein means to administer a compound of the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in several ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C <10% is considered a high anti-tumor activity level, with T/C (%)=median tumor volume of the treated/median tumor volume of the control×100.

In some embodiments, the treatment achieved by a compound of the invention is defined by reference to any of the following: partial response (PR), complete response (CR), overall response (OR), objective response rate (ORR), progression free survival (PFS), radiographic PFS, metastasis fee survival (MFS), disease free survival (DFS) and overall survival (05).

As used herein, the term “complete response” or “CR” means the disappearance of all signs of cancer (e.g., disappearance of all target lesions) in response to treatment. This does not always mean the cancer has been cured.

As used herein, the term “disease-free survival” (DFS) means the length of time after primary treatment for a cancer ends that the patient survives without any signs or symptoms of that cancer.

As used herein, the term “duration of response” (DoR) means the length of time that a tumor continues to respond to treatment without the cancer growing or spreading. Treatments that demonstrate improved DoR can produce a durable, meaningful delay in disease progression.

As used herein, the terms “objective response” and “overall response” refer to a measurable response, including complete response (CR) or partial response (PR). The term “overall response rate” (ORR) refers to the sum of the complete response (CR) rate and the partial response (PR) rate.

As used herein, the term “overall survival” (OS) means the length of time from either the date of diagnosis or the start of treatment for a disease, such as cancer, that patients diagnosed with the disease are still alive. OS is typically measured as the prolongation in life expectancy in patients who receive a certain treatment as compared to patients in a control group (i.e., taking either another drug or a placebo).

As used herein, the term “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

As used herein, the term “progression free survival” or “PFS” refers to the length of time during and after treatment during which the disease being treated (e.g., cancer) does not get worse. PFS, also referred to as “Time to Tumor Progression”, may include the amount of time patients have experienced a CR or PR, as well as the amount of time patients have experienced SD.

As used herein, the term “progressive disease” or “PD” refers to a cancer that is growing, spreading or getting worse. In some embodiments, PR refers to at least a 20% increase in the SLD of target lesions, taking as reference the smallest SLD recorded since the treatment started, or to the presence of one or more new lesions.

As used herein, the term “stable disease” (SD) refers to a cancer that is neither decreasing nor increasing in extent or severity.

As used herein, the term “sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

The anti-cancer effect of the method of treating cancer, including “objective response,” “complete response,” “partial response,” “progressive disease,” “stable disease,” “progression free survival,” “duration of response,” as used herein, may be defined and assessed by the investigators using RECIST v1.1 (Eisenhauer et al., New response evaluation criteria in solid tumours: Revised RECIST guideline (version 1.1), Eur J of Cancer, 2009; 45(2):228-47).

In some embodiments of each of the methods and uses described herein, the invention relates to neoadjuvant therapy, adjuvant therapy, first-line therapy, second-line therapy, second-line or later lines of therapy, or third-line or later lines of therapy. In each case as further described herein, the cancer may be localized, advanced or metastatic, and the intervention may occur at point along the disease continuum (i.e., at any stage of the cancer).

The treatment regimen for a compound of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-testy and the Wilcon on-test.

The terms “treatment regimen”, “dosing protocol” and “dosing regimen” may be used interchangeably to refer to the dose and timing of administration of any of the crystalline or amorphous forms of PF-07104091, as described in the present invention, alone or in combination with an additional anticancer agent.

“Ameliorating” means reducing to some extent or improving one or more symptoms upon treatment with a compound or drug, such as any of the crystalline or amorphous forms of PF-07104091, as described in the present invention, as compared to not administering the compound. “Ameliorating” also includes shortening or reduction in duration of a symptom. that is, reducing to some extent, preferably, eliminating a symptom.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). In frequent embodiments of the methods provided herein, the abnormal cell growth is cancer.

Abnormal cell growth includes the abnormal growth of: (1) tumors characterized by amplification or overexpression of CDK2; (2) tumors characterized by amplification or overexpression of CCNE1 and/or CCNE2; (3) tumors characterized by loss or Rb; and (4) tumors that are resistant to endocrine therapy, anti-HER2 targeted agents, CDK4/6 inhibition or chemotherapy (e.g., taxanes or platins).

In some embodiments, the methods and uses of the present invention may further comprise one or more additional anti-cancer agents. In some embodiments, the additional anti-cancer agent is selected from the group consisting of an anti-tumor agent, an anti-angiogenesis agent, a signal transduction inhibitor, and an antiproliferative agent. In some embodiments, the additional anti-cancer agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, and endocrine therapeutic agents, such as antiandrogens, androgen deprivation therapy (ADT), and antiestrogens. Additional anti-cancer agents may include small molecules therapeutics and pharmaceutically acceptable salts or solvates thereof, therapeutic antibodies, antibody-drug conjugates (ADCs), proteolysis targeting chimeras (PROTACs), or antisense molecules.

In some embodiments, the additional anti-cancer agent is an antiestrogen, wherein the antiestrogen is an aromatase inhibitor, a SERD, or a SERM. In some embodiments, the antiestrogen is an aromatase inhibitor. In some such embodiments, the aromatase inhibitor is selected from the group consisting of letrozole, anastrozole, and exemestane. In some such embodiments, the aromatase inhibitor is letrozole. In some embodiments, the antiestrogen is a SERD. In some such embodiments, the SERD is selected from the group consisting of fulvestrant, elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), and SHR9549 (Jiansu Hengrui Medicine). In some such embodiments, the SERD is fulvestrant. In some embodiments, the antiestrogen is a SERM. In some such embodiments, the SERM is selected from the group consisting of tamoxifen, raloxifene, toremifene, lasofoxifene, bazedoxifene and afimoxifene. In some such embodiments, the SERM is tamoxifen or raloxifene.

In some embodiments, the additional anti-cancer agent is an antiandrogen, such as abiraterone, apalutamide, bicalutamide, cyproterone, enzalutamide, flutamide, or nilutamide. In some embodiments, the method or use further comprises androgen deprivation therapy (ADT), e.g., a luteinizing hormone-releasing hormone (LHRH) agonist, a LHRH antagonist, a gonadotropin releasing hormone (GnRH) agonist or a GnRH antagonist.

In some embodiments, the methods and uses of the present invention further comprise one or more additional anti-cancer agents selected from the following:

Anti-angiogenesis agents include, for example, VEGF inhibitors, VEGFR inhibitors, TIE-2 inhibitors, PDGFR inhibitors, angiopoetin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors.

Signal transduction inhibitors include, for example, kinase inhibitors (e.g., inhibitors of tyrosine kinases, serine/threonine kinases or cyclin dependent kinases), proteasome inhibitors, PI3K/AKT/mTOR pathway inhibitors, phosphoinositide 3-kinase (PI3K) inhibitors, isocitrate dehydrogenase 1 and 2 (IDH1 and IDH2) inhibitors, B-cell lymphoma 2 (BCL2) inhibitors, neurotrophin receptor kinase (NTRK) inhibitors, Rearranged during Transfection (RET) inhibitors, Notch inhibitors, PARP inhibitors, Hedgehog pathway inhibitors, and selective inhibitors of nuclear export (SINE).

Examples of signal transduction inhibitors include, but are not limited to: acalabrutinib, afatinib, alectinib, alpelisib, axitinib, binimetinib, bortezomib, bosutinib, brigatinib, cabozantinib, carfilzomib, ceritinib, cobimetinib, copanlisib, crizotinib, dabrafenib, dacomitinib, dasatinib, duvelisib, enasidenib, encorafenib, entrectinib, erlotinib, gefitinib, gilteritinib, glasdegib, ibrutinib, idelalisib, imatinib, ipatasertib, ivosidenib, ixazomib, lapatinib, larotrectinib, lenvatinib, lorlatinib, midostaurin, neratinib, nilotinib, niraparib, olaparib, osimertinib, pazopanib, ponatinib, regorafenib, rucaparib, ruxolitinib, sonidegib, sorafenib, sunitinib, talazoparib, trametinib, vandetanib, vemurafenib, venetoclax, and vismodegib, or pharmaceutically acceptable salts and solvates thereof.

Antineoplastic agents include, for example, alkylating agents, platinum coordination complexes, cytotoxic antibiotics, antimetabolies, biologic response modifiers, histone deacetylate (HDAC) inhibitors, hormonal agents, monoclonal antibodies, growth factor inhibitors, taxanes, topoisomerase inhibitors, Vinca alkaloids and miscellaneous agents.

Alkylating agents include: altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa, and trabectedin.

Platinum coordination complexes (also referred to herein as “platinum agents”) include: carboplatin, cisplatin, and oxaliplatin.

Cytotoxic antibiotics include: bleomycin, dactinomycin, daunorubicin, doxorubicin, epirubicin, idarubicin, mitomycin, mitoxantrone, plicamycin, and valrubicin.

Antimetabolites include: antifolates, such as methotrexate, pemetrexed, pralatrexate, and trimetrexate; purine analogues, such as azathioprine, cladribine, fludarabine, mercaptopurine, and thioguanine; and pyrimidine analogues such as azacitidine, capecitabine, cytarabine, decitabine, floxuridine, fluorouracil, gemcitabine, and trifluridine/tipracil.

Biologic response modifiers include: aldesleukin (IL-2), denileukin diftitox, and interferon gamma.

Histone deacetylase inhibitors include belinostat, panobinostat, romidepsin, and vorinostat.

Hormonal agents include antiandrogens, antiestrogens, gonadotropin releasing hormone (GnRH) analogues and peptide hormones. Examples of antiestrogens include: aromatase inhibitors, such as letrozole, anastrozole, and exemestane; SERDs, such as fulvestrant, elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), SHR9549 (Jiansu Hengrui Medicine); and serMs, such as tamoxifen, raloxifene, toremifene, lasofoxifene, bazedoxifene, afimoxifene. Examples of GnRH analogues include: degarelix, goserelin, histrelin, leuprolide, and triptorelin. Examples of peptide hormones include: lanreotide, octreotide, and pasireotide. Examples of antiandrogens include: abiraterone, apalutamide, bicalutamide, cyproterone, enzalutamide, flutamide, and nilutamide, and pharmaceutically acceptable salts and solvates thereof.

Monoclonal antibodies include: alemtuzumab, atezolizumab, avelumab, bevacizumab, blinatumomab, brentuximab, cemiplimab, cetuximab, daratumumab, dinutuximab, durvalumab, elotuzumab, gemtuzumab, inotuzumab ozogamicin, ipilimumab, mogamulizumab, moxetumomab pasudotox, necitumumab, nivolumab, ofatumumab, olaratumab, panitumumab, pembrolizumab, pertuzumab, ramucirumab, rituximab, tositumomab, and trastuzumab.

Taxanes include: cabazitaxel, docetaxel, paclitaxel and paclitaxel albumin-stabilized nanoparticle formulation (Nab-paclitaxel).

Topoisomerase inhibitors include: etoposide, irinotecan, teniposide, and topotecan.

Vinca alkaloids include: vinblastine, vincristine, and vinorelbine, and pharmaceutically acceptable salts thereof.

Miscellaneous antineoplastic agents include: asparaginase (pegaspargase), bexarotene, eribulin, everolimus, hydroxyurea, ixabepilone, lenalidomide, mitotane, omacetaxine, pomalidomide, tagraxofusp, telotristat, temsirolimus, thalidomide, and venetoclax.

In some embodiments, the additional anti-cancer agent is selected from the group consisting of: abiraterone acetate; acalabrutinib; ado-trastuzumab emtansine; afatinib dimaleate; afimoxifene; aldesleukin; alectinib; alemtuzumab; alpelisib; am ifostine; anastrozole; apalutamide; aprepitant; arsenic trioxide; asparaginase erwinia chrysanthemi; atezolizumab; avapritinib; avelumab; axicabtagene ciloleucel; axitinib; azacitidine; AZD9833 (AstraZeneca); AZD9496 (AstraZeneca); bazedoxifene; belinostat; bendamustine hydrochloride; bevacizumab; bexarotene; bicalutamide; binimetinib; bleomycin sulfate; blinatumomab; bortezomib; bosutinib; brentuximab vedotin; brigatinib; cabazitaxel; cabozantinib-s-malate; calaspargase pegol-mknl; capecitabine; aplacizumab-yhdp; capmatinib hydrochloride; carboplatin; carfilzomib; carmustine; cemiplimab-rwlc; ceritinib; cetuximab; chlorambucil; cisplatin; cladribine; clofarabine; cobimetinib; copanlisib hydrochloride; crizotinib; cyclophosphamide; cytarabine; D-0502 (Inventisbio); dabrafenib mesylate; dacarbazine; dacomitinib; dactinomycin; daratumumab; daratumumab and hyaluronidase-fihj; darbepoetin alfa; darolutamide; dasatinib; daunorubicin hydrochloride; decitabine; defibrotide sodium; degarelix; denileukin diftitox; denosumab; dexamethasone; dexrazoxane hydrochloride; dinutuximab; docetaxel; doxorubicin hydrochloride; durvalumab; duvelisib; elacestrant; elotuzumab; eltrombopag olamine; emapalumab-lzsg; enasidenib mesylate; encorafenib; enfortumab vedotin-ejfv; entrectinib; enzalutamide; epirubicin hydrochloride; epoetin alfa; erdafitinib; eribulin mesylate; erlotinib hydrochloride; etoposide; etoposide phosphate; everolimus; exemestane; fam-trastuzumab deruxtecan-nxki; fedratinib hydrochloride; filgrastim; fludarabine phosphate; fluorouracil; flutamide; fostamatinib disodium; fulvestrant; gefitinib; gemcitabine hydrochloride; gemtuzumab ozogamicin; gilteritinib fumarate; glasdegib maleate; glucarpidase; goserelin acetate; granisetron; granisetron hydrochloride; hydroxyurea; ibritumomab tiuxetan; ibrutinib; idarubicin hydrochloride; idelalisib; ifosfamide; imatinib mesylate; imiquimod; inotuzumab ozogamicin; interferon alfa-2b recombinant; iobenguane I-131; ipatasertib; ipilimumab; irinotecan hydrochloride; isatuximab-irfc; ivosidenib; ixabepilone; ixazomib citrate; lanreotide acetate; lapatinib ditosylate; larotrectinib sulfate; lasofoxifene; lenalidomide; lenvatinib mesylate; letrozole; leucovorin calcium; leuprolide acetate; lomustine; lorlatinib; LSZ102 (Novartis); lurbinectedin; LY3484356 (Lilly); megestrol acetate; melphalan; melphalan hydrochloride; mercaptopurine; methotrexate; midostaurin; mitomycin ; mitoxantrone hydrochloride; mogamulizumab-kpkc; moxetumomab pasudotox-tdfk; necitum um ab; nelarabine; neratinib maleate; nilotinib; nilutamide; niraparib tosylate monohydrate; nivolumab; obinutuzumab; ofatumumab; olaparib; omacetaxine mepesuccinate; ondansetron hydrochloride; osimertinib mesylate; oxaliplatin; paclitaxel; paclitaxel albumin-stabilized nanoparticle formulation; paliferm in; palonosetron hydrochloride; pamidronate disodium; panitumumab; panobinostat; pazopanib hydrochloride; pegaspargase; pegfilgrastim; peginterferon alfa-2b; pembrolizumab; pemetrexed disodium; pemigatinib; pertuzumab; pexidartinib hydrochloride; plerixafor; polatuzumab vedotin-piiq; pomalidomide; ponatinib hydrochloride; pralatrexate; prednisone; procarbazine hydrochloride; propranolol hydrochloride; radium 223 dichloride; raloxifene hydrochloride; ramucirumab; rasburicase; ravulizumab-cwvz; recombinant interferon alfa-2b; regorafenib; RG6171 (Roche); rintodestrant; ripretinib; rituximab; rolapitant hydrochloride; romidepsin; romiplostim; rucaparib camsylate; ruxolitinib phosphate; sacituzumab govitecan-hziy; SAR439859 (Sanofi); selinexor; selpercatinib; selumetinib sulfate; SHR9549 (Jiansu Hengrui Medicine); siltuximab; sipuleucel-t; sonidegib; sorafenib tosylate; tagraxofusperzs; talazoparib tosylate; talimogene laherparepvec; tamoxifen citrate; tazemetostat hydrobromide; temozolomide; temsirolimus; thalidomide; thioguanine; thiotepa; tisagen lecleucel; tocilizumab; topotecan hydrochloride; torem ifene; trabected in; trametinib; trastuzumab; trastuzumab and hyaluronidase-oysk; trifluridine and tipiracil hydrochloride; tucatinib; uridine triacetate; valrubicin; vandetanib; vemurafenib; venetoclax; vinblastine sulfate; vincristine sulfate; vinorelbine tartrate; vismodegib; vorinostat; zanubrutinib ; ziv-aflibercept; ZN-c5 (Zentalis); and zoledronic acid; or free base, pharmaceutically acceptable salt (including an alternative salt forms to the salts named above), or solvate forms of the foregoing; or combinations thereof.

The terms “cancer” or “cancerous” refer to or describe malignant and/or invasive growth or tumor caused by abnormal cell growth. As used herein “cancer” refers to solid tumors named for the type of cells that form them, as well as cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

The efficacy of the methods and uses described herein in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents, such as PD-1 or PD-L1 antagonists and the like. The methods and uses of the current invention may further comprise one or more additional anti-cancer agents.

Administration of crystalline or amorphous forms of the invention may be affected by any method that enables delivery of the compound to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, the crystalline or amorphous form of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the solid form and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compounds or pharmaceutical compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed solid form or pharmaceutical composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein

The dosage of the crystalline or amorphous form of the invention is typically in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day. The dosage may be administered as a single dose (QD), or optionally may be subdivided into smaller doses, suitable for BID (twice daily), TID (three times daily) or QID (four times daily) administration. The dosage regimen may be adjusted to provide the optimal therapeutic response. For example, the dose may be proportionally reduced or increased as indicated by the exigencies of the therapeutic situation, including temporary or permanent dose reductions if required to ameliorate or prevent side effects.

Repetition of the administration or dosing regimens, or adjustment of the administration or dosing regimen may be conducted as necessary to achieve the desired treatment. A “continuous dosing schedule” as used herein is an administration or dosing regimen without dose interruptions, e.g., without days off treatment. Repetition of 21 or 28 day treatment cycles without dose interruptions between the treatment cycles is an example of a continuous dosing schedule.

In some embodiments, the crystalline or amorphous form of PF-07104091 is administered at a daily dosage of from about 1 mg to about 1000 mg per day. In some embodiments, the crystalline or amorphous form of the invention is administered at a daily dosage from about 10 mg to about 500 mg per day, and in some embodiments, it is administered at a dosage of from about 25 mg to about 300 mg per day. In some embodiments it is administered at dosages of about 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 260, 270, 275, 280, 290, 300, 325, 350, 375, 400, 425, 450, 475 or 500 mg on a QD, BID, TID or QID schedule.

Repetition of the administration or dosing regimens, or adjustment of the administration or dosing regimen may be conducted as necessary to achieve the desired treatment. An “intermittent dosing schedule” refers to an administration or dosing regimen that includes a period of dose interruption, e.g., days off treatment. Repetition of 14 or 21 day treatment cycles with a 7 day treatment interruption between the treatment cycles is an example of an intermittent dosing schedule. Such schedules, with 2 or 3 weeks on treatment and 1 week off treatment, are sometimes referred to as a 2/1-week or 3/1-week treatment cycle, respectively. Alternatively, intermittent dosing may comprise a 7 day treatment cycle, with 5 days on treatment and 2 days off treatment.

A “continuous dosing schedule” as used herein is an administration or dosing regimen without dose interruptions, e.g., without days off treatment. Repetition of 21 or 28 day treatment cycles without dose interruptions between the treatment cycles is an example of a continuous dosing schedule.

In some embodiments, any of the crystalline or amorphous forms of PF-07104091 described herein is administered in an intermittent dosing schedule. In other embodiments, any of the crystalline or amorphous forms of PF-07104091 described herein is administered in a continuous dosing schedule.

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

In one embodiment, the invention relates to a pharmaceutical composition comprising anhydrous crystalline PF-07104091 (Form 2), and a pharmaceutically acceptable carrier or excipient.

In one embodiment, the invention relates to a pharmaceutical composition comprising crystalline PF-07104091 monohydrate (Form 3), and a pharmaceutically acceptable carrier or excipient.

In one embodiment, this invention relates to a pharmaceutical composition comprising amorphous PF-07104091 (Form 4) and a pharmaceutically acceptable carrier or excipient.

In one embodiment, the invention relates to a pharmaceutical composition comprising anhydrous crystalline PF-07104091 (Form 5), and a pharmaceutically acceptable carrier or excipient.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid pharmaceutical compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Pharmaceutical compositions of the present invention may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 19th Edition (1995).

Any of the crystalline or amorphous forms of the invention described herein may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Any of the crystalline or amorphous forms of the invention described herein may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the crystalline or amorphous form of PF-07104091 may make up from 1 weight % (wt %) to 80 wt % of the dosage form, more typically from 5 wt % to 60 wt % of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets may contain from about 1 wt % to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Capsules (made, for example, from gelatin or HPMC), blisters and cartridges for use in an inhaler or insufflator may be formulated to contain a powder mix of the therapeutic agent, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al, Current Status of Drug Delivery Technologies and Future Directions, Pharmaceutical Technology On-line, (2001) 25:1-14. The use of chewing gum to achieve controlled release is described in WO 00/35298. The disclosures of these references are incorporated herein by reference in their entireties.

Any of the crystalline or amorphous forms of PF-07104091 described herein may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of therapeutic agents used in the preparation of parenteral solutions may potentially be increased by the use of appropriate formulation techniques, such as the incorporation of solubility-enhancing agents.

The crystalline and amorphous forms of PF-07104091 described herein may be in the form of a kit suitable for administration of the pharmaceutical composition. Such kits may comprise the active agent in the form of a pharmaceutical composition, which pharmaceutical composition comprises an active agent, or a pharmaceutically acceptable salt or solvate thereof, and a pharmaceutically acceptable carrier. The kit may contain means for separately retaining the pharmaceutical composition, such as a container, divided bottle, or divided foil packet. An example of such a kit is the familiar blister pack used for the packaging of tablets, capsules and the like. To assist in compliance, the kit typically includes directions for administration and may be provided with a memory aid. The kit may further comprise other materials that may be useful in administering the medicament, such as diluents, filters, IV bags and lines, needles and syringes, and the like.

In some preferred embodiments, the invention provides one or more of embodiments E1 to E41:
E1. A crystalline form of (1R,3S)-3-[3-(({[3-(methoxymethyl)-1-methyl-1H-pyrazol-5-yl]carbonyl}amino)-1H-pyrazol-5-yl]cyclopentyl propan-2-ylcarbamate (PF-07104091) monohydrate (Form 3), having a powder X-ray diffraction (PXRD) pattern comprising peaks at 2θ values measured using CuKcx, radiation of: 8.4, 10.1 and 21.5 °2θ±0.2 °2θ.
E2. The crystalline form of embodiment E1, having a PXRD pattern further comprising a peak at a 2θ value measured using CuKα, radiation of: 16.9 20 2θ±0.2 °2θ.
E3. The crystalline form of embodiment E1 or E2, having a PXRD pattern further comprising a peak at a 2θ value measured using CuKα, radiation of: 27.0 °2θ±0.2 °2θ.
E4. The crystalline form of any one of embodiments E1 to E3, having a Raman spectrum comprising a wavenumber (cm⁻¹) value of: 1657 cm⁻¹+2 cm⁻¹.
E5. The crystalline form of embodiment E4, having a Raman spectrum further comprising a wavenumber (cm⁻¹) value of: 1595 cm⁻¹±2 cm⁻¹.
E6. The crystalline form of embodiment E4 or E5, having a Raman spectrum further comprising a wavenumber (cm⁻¹) value of: 1408 cm⁻¹±2 cm⁻¹.
E7. The crystalline form of any one of embodiments E1 to E6, having a ¹³C solid state NMR spectrum comprising one, two or three resonance (ppm) values selected from the group consisting of: 25.2, 37.5 and 159.3 ppm±0.2 ppm.
E8. The crystalline form of embodiment E7, having a ¹³C solid state NMR spectrum further comprising resonance (ppm) values of: 151.9 and 152.5 ppm±0.2 ppm.
E9. A crystalline form PF-07104091 monohydrate (Form 3), having a Raman spectrum comprising wavenumber (cm⁻¹) values of: 1657, 1595 and 1408 cm⁻¹±2 cm⁻¹.

E10. A crystalline form of PF-07104091 monohydrate (Form 3), having a ¹³C solid state NMR spectrum comprising resonance (ppm) values of: 25.2 and 37.5 ppm±0.2 ppm; and optionally further comprising a resonance (ppm) value of: 159.3 ppm±0.2 ppm.
E11. The crystalline form of embodiment E10, having a PXRD pattern comprising peaks at 2θ values measured using CuKα radiation of: 8.4 and 10.1 °2θ±0.2 °2θ.

E12. The crystalline form of embodiment E10 or E11, having a Raman spectrum comprising a wavenumber (cm⁻¹) value of: 1657 cm⁻¹±2 cm⁻¹.
E13. A crystalline form of PF-07104091 monohydrate (Form 3), having:
(a) a PXRD pattern comprising peaks at 2θ values measured using CuKα radiation of: 8.4, 10.1 and 21.5 °2θ±0.2 °2θ;
(b) a Raman spectrum comprising wavenumber (cm⁻¹) values of: 1657, 1595 and 1408 cm⁻¹+2 cm⁻¹; or
(c) a ¹³C solid state NMR spectrum comprising resonance (ppm) values of: 25.2, 37.5 and 159.3 ppm±0.2 ppm;
or any combination of two or more of (a), (b), and (c).
E14. A crystalline form of PF-07104091 monohydrate (Form 3), having:
(a) a PXRD pattern comprising peaks at 2θ values measured using CuKα radiation of: 8.4 and 10.1 °2θ±0.2 °2θ; and optionally further comprising a peak at a 2θ value measured using CuKα radiation of: 21.5 °2θ±0.2 °2θ;
(b) a Raman spectrum comprising a wavenumber (cm⁻¹) value of: 1657 cm⁻¹±2 cm⁻¹; and optionally further comprising wavenumber (cm⁻¹) values of: 1595 and 1408 cm⁻¹±2 cm⁻¹; or
(c) a ¹³C solid state NMR spectrum comprising resonance (ppm) values of: 25.2 and 37.5 ppm±0.2 ppm; and optionally further comprising a resonance (ppm) value of: 159.3 ppm±0.2 ppm;
or any combination of two or more of (a), (b), and (c).
E15. The crystalline form of any one of embodiments E1 to E14, wherein the crystalline form is substantially pure PF-07104091 monohydrate (Form 3).
E16. An anhydrous crystalline form of PF-07104091 (Form 2), having a PXRD pattern comprising peaks at 2θ values measured using CuKα radiation of: 9.8, 13.3 and 17.4 °2θ±0.2 °2θ.
E17. The crystalline form of embodiment E16, having a PXRD pattern further comprising a peak at a 2θ value measured using CuKα radiation of: 4.2 °2θ±0.2 °2θ.
E18. The crystalline form of embodiment E16 or E17, having a PXRD pattern further comprising a peak at a 2θ value measured using CuKα radiation of: 7.5 20 2θ±0.2 °2θ.
E19. The crystalline form of any one of embodiments E16 to E18, having a Raman spectrum comprising a wavenumber (cm⁻¹) value of: 1691 cm⁻¹±2 cm⁻¹.
E20. The crystalline form of embodiment E19, having a Raman spectrum further comprising a wavenumber (cm⁻¹) value of: 1582 cm⁻¹+2 cm⁻¹.
E21. The crystalline form of embodiment E19 or E20, having a Raman spectrum further comprising a wavenumber (cm⁻¹) value of: 996 cm⁻¹±2 cm⁻¹.
E22. The crystalline form of any one of embodiments E16 to E21, having a ¹³C solid state NMR spectrum comprising one, two or three resonance (ppm) values selected from the group consisting of: 24.1, 39.8 and 41.6 ppm±0.2 ppm.
E23. The crystalline form of embodiment E22, having a ¹³C solid state NMR spectrum further comprising resonance (ppm) values of: 21.8 and 138.2 ppm±0.2 ppm.
E24. An anhydrous crystalline form of PF-07104091 (Form 2), having a Raman spectrum comprising wavenumber (cm⁻¹) values of: 1691, 1582 and 996 cm⁻¹±2 cm⁻¹.
E25. An anhydrous crystalline form of PF-07104091 (Form 2), having a ¹³C solid state NMR spectrum comprising resonance (ppm) values of: 24.1, 39.8 and 41.6 ppm±0.2 ppm.
E26. The crystalline form of embodiment E25, having a PXRD pattern comprising peaks at 2θ values measured using CuKα radiation of: 9.8 and 13.3 °2θ±0.2 °2θ.
E27. The crystalline form of embodiment E25 or E26, having a Raman spectrum comprising a wavenumber (cm⁻¹) value of: 1691 cm⁻¹±2 cm⁻¹.
E28. An anhydrous crystalline form of PF-07104091 (Form 2), having:
(a) a PXRD pattern comprising peaks at 2θ values measured using CuKα radiation of: 9.8, 13.3 and 17.4 °2θ±0.2 °2θ;
(b) a Raman spectrum comprising wavenumber (cm⁻¹) values of: 1691, 1582 and 996 cm⁻¹+2 cm⁻¹; or
(c) a ¹³C solid state NMR spectrum comprising resonance (ppm) values of: 24.1, 39.8 and 41.6 ppm±0.2 ppm;
or any combination of two or more of (a), (b), and (c).
E29. An anhydrous crystalline form of PF-07104091 (Form 2), having:
(a) a PXRD pattern comprising peaks at 2θ values measured using CuKα radiation of: 9.8 and 13.3 20 2θ±0.2 °2θ; and optionally further comprising a peak at a 2θ value measured using CuKα radiation of: 17.4 °2θ±0.2 °2θ;
(b) a Raman spectrum comprising a wavenumber (cm⁻¹) value of: 1691 cm⁻¹±2 cm ⁻¹, and optionally further comprising wavenumber (cm⁻¹) values of: 1582 and 996 cm⁻¹±2 cm⁻¹, or
(c) a ¹³C solid state NMR spectrum comprising resonance (ppm) values of: 24.1, 39.8 and 41.6 ppm±0.2 ppm;
or any combination of two or more of (a), (b), and (c).
E30. The crystalline form of any one of embodiments E16 to E29, wherein the crystalline form is substantially pure PF-07104091 (Form 2).
E31. An anhydrous crystalline form of PF-07104091 (Form 5), having a PXRD pattern comprising three or more peaks at 2θ values measured using CuKα radiation selected from the group consisting of: 10.2, 12.4, 15.4, 17.2, 17.9, 19.8, 21.6, 22.5, 23.7 and 26.2 20 2θ±0.2 °2θ.
E32. A pharmaceutical composition comprising the crystalline form of any one of embodiments E1 to E31, and a pharmaceutically acceptable carrier or excipient.
E33. A method of treating cancer in a subject in need thereof, comprising administering to the subject a therapeutically effective amount of the crystalline form of any one of embodiments E1 to E31.
E34. The method of embodiment E33, wherein the cancer is selected from the group consisting of breast cancer, prostate cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.
E35. An amorphous form of PF-07104091 (Form 4).
E36. The amorphous form of embodiment E35, having a PXRD pattern comprising a broad peak at diffraction angles (2θ) measured using CuKα radiation from about 5 to about 35 °2θ±0.2 °2θ.
E37. The amorphous form of embodiment E35 or E36, having a PXRD pattern essentially the same as in FIG. 4.
E38. The amorphous form of any one of embodiments E35 to E37, having a glass transition temperature (T_{g}) of 59.8±5° C.
E39. A pharmaceutical composition comprising the amorphous form of any one of embodiments E35 to E38, and a pharmaceutically acceptable carrier or excipient.
E40. A method of treating cancer in a subject in need thereof, comprising administering to the subject a therapeutically effective amount of the amorphous form of any one of embodiments E35 to E38.
E41. The method of embodiment E40, wherein the cancer is selected from the group consisting of breast cancer, prostate cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.

### EXAMPLES

The examples and preparations provided below further illustrate and exemplify aspects and embodiments of the invention. It is to be understood that the scope of the present invention is not limited by the scope of the following examples.

### Example 1

### Instrumental Methods

### General Method A. Powder X-ray Diffraction (PXRD)

### Instrument Method:

Powder X-ray diffraction analysis was conducted using a Bruker AXS D8 Endeavor diffractometer equipped with a Cu radiation source. Diffracted radiation was detected by a LYNXEYE XE T detector with motorized slits. The X-ray tube voltage and amperage were set at 40 kV and 40 mA respectively. Data was collected in the Theta-Theta goniometer in a locked couple scan at Cu K-alpha wavelength (CuKα λ=1.5418 Å) from 3.0 to 40.0 degrees 2-Theta with an increment of 0.01 degrees, using a scan speed of 1.0 seconds per step. The antiscatter screen was set to a fixed distance of 1.5 mm. Samples were prepared by placement in a silicon low background sample holder. Samples were rotated at 15 rotations per minute during collection. Data were collected using Bruker DIFFRAC Plus software.

### Peak Picking Method:

Data analysis was performed using Bruker DIFFRAC Plus software (version 5.0.0). The PXRD data file was not processed prior to peak searching. The peak search algorithm in the EVA software was applied to make preliminary peak assignments using a threshold value of 1. To ensure validity, adjustments were manually made; the output of automated assignments was visually checked, and peak positions were adjusted to the peak maximum. Peaks with relative intensity of ≥3% were generally chosen. The peaks which were not resolved or were consistent with noise were not selected. A typical error associated with the peak position from PXRD stated in USP up to +/−0.2° 2-Theta (USP-941).

### General Method B. Raman Spectroscopy

### Instrument Method:

Raman spectra were collected using a Thermo Scientific iS50 FT-Raman accessory attached to the FT-IR bench. A CaF2 beam splitter is utilized in the FT-Raman configuration. The spectrometer is equipped with a 1064 nm diode laser and a room temperature InGaAs detector. Prior to data acquisition, instrument performance and calibration verifications were conducted using polystyrene. Samples were analyzed in glass NMR tubes, as tablets or in a suitable sample holder held static during data collection. The spectra were collected using between 0.1 and 0.5 W of laser power and 512 co-added scans. The collection range was 3700-100 cm⁻¹. The API spectra were recorded using 2 cm⁻¹ resolution, and Happ-Genzel apodization was utilized for all of the spectra. Multiple spectra were recorded, and the reported spectrum is representative of two spots.

### Peak Picking Method:

The intensity scale was normalized to 1 prior to peak picking. Peaks were manually identified using the Thermo Nicolet Omnic 9.7.46 software. Peak position was picked at the peak maximum, and peaks were only identified as such, if there was a slope on each side; shoulders on peaks were not included. For neat Form 3 API an absolute threshold of 0.012 with a sensitivity of 75 was utilized during peak picking. For neat Form 2 API an absolute threshold of 0.04 with a sensitivity of 75 was utilized during peak picking. Peaks with normalized peak intensity between (1-0.75), (0.74-0.30), (0.29-0) were labeled as strong, medium and weak, respectively. The relative peak intensity values are also illustrated in this report.

General Method C. ¹³C Solid State NMR (ssNMR) Spectroscopy:

### Instrument Method:

Solid state NMR (ssNMR) analysis was conducted on a CPMAS probe positioned into a Bruker-BioSpin Avance III 500 MHz (1 H frequency) NMR spectrometer. Material was packed into a 4 mm rotor. A magic angle spinning rate of 15.0 kHz was used.

¹³C ssNMR spectra were collected using a proton decoupled cross-polarization magic angle spinning (CPMAS) experiment. A phase modulated proton decoupling field of 80-90 kHz was applied during spectral acquisition. The cross-polarization contact time was set to 2 ms. Recycle delay of 4.5 seconds, 3.9 seconds, 4.5 seconds, and 2.4 seconds were used in experiments on Form 1, Form 2, Form 3, and Form 5, respectively. The number of scans was adjusted to obtain an adequate signal to noise ratio, with 768 or 1024 scans being collected for each API. The ¹³C chemical shift scale was referenced using a ¹³C CPMAS experiment on an external standard of crystalline adamantane, setting its up-field resonance to 29.5 ppm (as determined from neat TMS).

### Peak Picking Method:

Automatic peak picking was performed using Bruker-BioSpin TopSpin version 3.6 software. Generally, a threshold value of 5% relative intensity was used for preliminary peak selection. The output of the automated peak picking was visually checked to ensure validity and adjustments were manually made, if necessary. Although specific solid state NMR peak values are reported herein there does exist a range for these peak values due to differences in instruments, samples, and sample preparation. This is common practice in the art of solid state NMR because of the variation inherent in peak positions. A typical variability for ¹³C chemical shift x-axis value is on the order of plus or minus 0.2 ppm for a crystalline solid. The solid state NMR peak heights reported herein are relative intensities. Solid state NMR intensities can vary depending on the actual setup of the CPMAS experimental parameters and the thermal history of the sample.

### Example 2

### Preparation of Anhydrous Crystalline PF-07104091 (Form 2)

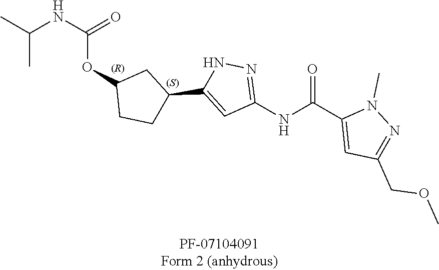

Anhydrous crystalline PF-07104091 (Form 2) was prepared by dissolving PF-07104091 monohydrate (Form 1) (prepared as described in U.S. Pat. No. 11,014,911) in 50:50% v/v methyl isobutyl ketone: heptane at ˜80° C. The solution was then removed from the heat and allowed to cool to room temperature. The resulting solids were collected by filtration, rinsed with heptane, and dried under vacuum to provide crystalline PF-07104091 (Form 2), which was confirmed by elemental analysis to be an anhydrous free form.

PF-07104091 (Form 2) was also obtained by crystallization from other solvents (e.g., ethyl acetate, cyclohexane, or mixtures thereof), as shown by PXRD analysis. In some cases, small amounts of residual solvent was detectable by ssNMR, likely due to solvent trapped within crystal lattice defects during crystallization of the anhydrous form.

Differential Scanning calorimetry (DSC) showed a melting endotherm (confirmed by melting point apparatus) with an onset temperature of ˜113° C.

PF-07104091 (Form 2) was found to be slightly hygroscopic by a moisture sorption (DVS) study: ˜0.7% mass gain at 60% RH; ˜1% mass gain at 75% RH; and ˜1.6% mass gain at 90% RH. PXRD of the post moisture sorption material showed no change in solid form.

Thermogravimetric Analysis (TGA) on an authentic sample of Form 2 crystallized from acetone:cyclohexane (1:2.1) showed ˜1% total weight loss that was confirmed to be residual solvent (cyclohexane) by solution NMR.

TABLE 1
PXRD peak list for PF-07104091 (Form 2)

| Angle (2-theta °) | Relative | Angle (2-theta °) | Relative |
|---|---|---|---|
| ±0.2 °2θ | Intensity (%) | ±0.2 °2θ | Intensity (%) |
| 3.6 | 6.1 | 18.3 | 14.9 |
| 4.1 | 100.0 | 18.6 | 6.0 |
| 4.2 | 57.9 | 18.9 | 4.0 |
| 5.7 | 5.2 | 19.4 | 13.0 |
| 6.0 | 5.1 | 19.7 | 30.3 |
| 6.8 | 5.9 | 20.6 | 6.8 |
| 7.2 | 26.0 | 21.3 | 8.1 |
| 7.5 | 40.7 | 21.5 | 13.2 |
| 8.2 | 3.5 | 21.6 | 15.4 |
| 8.5 | 4.0 | 21.9 | 7.9 |
| 9.4 | 5.3 | 22.1 | 3.1 |
| 9.8 | 87.6 | 22.3 | 3.1 |
| 11.0 | 23.3 | 22.6 | 9.7 |
| 11.5 | 29.6 | 24.0 | 4.2 |
| 11.7 | 4.8 | 24.4 | 5.3 |
| 12.8 | 17.7 | 24.9 | 3.0 |
| 13.3 | 75.5 | 25.9 | 9.3 |
| 13.7 | 6.0 | 26.7 | 9.2 |
| 14.5 | 45.3 | 27.3 | 3.1 |
| 14.9 | 7.3 | 27.7 | 3.0 |
| 15.5 | 8.8 | 28.0 | 3.7 |
| 15.9 | 35.1 | 29.4 | 5.7 |
| 16.4 | 6.0 | 31.4 | 5.5 |
| 17.4 | 45.6 | | |

TABLE 2
Raman peak list for PF-07104091 (Form 2)

| Peak position | | Peak position | |
|---|---|---|---|
| cm⁻¹ | Normalized | cm⁻¹ | Normalized |
| ±2 cm⁻¹ | intensity | ±2 cm⁻¹ | intensity |
| 100 | 0.99 | 1170 | 0.23 |
| 163 | 0.55 | 1213 | 0.12 |
| 174 | 0.37 | 1242 | 0.26 |
| 207 | 0.39 | 1268 | 0.29 |
| 301 | 0.27 | 1292 | 0.25 |
| 331 | 0.26 | 1352 | 0.33 |
| 342 | 0.26 | 1365 | 0.31 |
| 354 | 0.28 | 1386 | 0.56 |
| 367 | 0.29 | 1404 | 0.55 |
| 401 | 0.23 | 1461 | 0.62 |
| 421 | 0.25 | 1471 | 0.63 |
| 449 | 0.30 | 1488 | 0.55 |
| 459 | 0.23 | 1551 | 0.52 |
| 477 | 0.20 | 1582 | 0.30 |
| 511 | 0.19 | 1591 | 0.32 |
| 536 | 0.17 | 1621 | 0.10 |
| 553 | 0.14 | 1676 | 0.46 |
| 593 | 0.12 | 1691 | 1.00 |
| 616 | 0.13 | 1750 | 0.06 |
| 630 | 0.14 | 1764 | 0.06 |
| 641 | 0.16 | 1791 | 0.05 |
| 661 | 0.18 | 1797 | 0.05 |
| 688 | 0.13 | 1835 | 0.05 |
| 696 | 0.13 | 1850 | 0.04 |
| 717 | 0.19 | 1896 | 0.04 |
| 726 | 0.18 | 1929 | 0.04 |
| 759 | 0.14 | 2696 | 0.04 |
| 785 | 0.14 | 2722 | 0.05 |
| 802 | 0.12 | 2740 | 0.04 |
| 818 | 0.13 | 2760 | 0.04 |
| 845 | 0.11 | 2790 | 0.04 |
| 872 | 0.28 | 2829 | 0.16 |
| 901 | 0.18 | 2874 | 0.33 |
| 913 | 0.12 | 2896 | 0.44 |
| 933 | 0.14 | 2945 | 0.64 |
| 947 | 0.16 | 2971 | 0.53 |
| 956 | 0.15 | 2987 | 0.48 |
| 977 | 0.12 | 3107 | 0.07 |
| 996 | 0.16 | 3128 | 0.10 |
| 1004 | 0.23 | 3156 | 0.05 |
| 1017 | 0.15 | 3192 | 0.05 |
| 1036 | 0.26 | 3208 | 0.04 |
| 1052 | 0.17 | 3218 | 0.04 |
| 1077 | 0.21 | 3233 | 0.05 |
| 1091 | 0.19 | 3298 | 0.04 |
| 1107 | 0.15 | 3334 | 0.11 |
| 1133 | 0.14 | 3392 | 0.04 |

TABLE 3
¹³C ssNMR peak list for PF-07104091 (Form 2)

| ¹³C Chemical | Relative | ¹³C Chemical | Relative |
|---|---|---|---|
| Shifts ppm | Intensity | Shifts ppm | Intensity |
| ±0.2 ppm | (%) | ±0.2 ppm | (%) |
| 14.3 † | 3 | 67.6 | 31 |
| 21.8 | 37 | 67.8 | 33 |
| 22.5 | 67 | 68.4 | 30 |
| 23.0 | 45 | 70.1 | 26 |
| 23.4 | 60 | 74.0 | 32 |
| 24.1 | 100 | 75.8 | 60 |
| 29.3 | 20 | 76.0 | 54 |
| 29.7 | 18 | 76.8 | 33 |
| 30.3 | 36 | 92.3 | 23 |
| 30.6 | 49 | 92.9 | 19 |
| 32.6 | 13 | 94.4 | 36 |
| 33.8 | 31 | 95.2 | 24 |
| 34.0 | 32 | 104.8 | 24 |
| 34.8 | 54 | 106.9 | 42 |
| 35.6 | 59 | 107.2 | 28 |
| 36.0 | 50 | 108.3 | 21 |
| 36.9 | 92 | 134.6 | 16 |
| 38.7 | 36 | 135.3 | 18 |
| 39.8 | 76 | 135.9 | 18 |
| 40.7 | 33 | 137.5 | 16 |
| 41.6 | 27 | 138.2 | 17 |
| 42.6 | 24 | 145.0 | 20 |
| 43.1 | 49 | 146.2 | 45 |
| 43.6 | 37 | 146.7 | 36 |
| 45.2 | 21 | 146.9 | 38 |
| 52.7 † | 2 | 147.6 | 55 |
| 56.6 | 45 | 149.0 | 62 |
| 56.7 | 45 | 150.5 | 12 |
| 58.7 | 34 | 156.2 | 52 |
| 59.3 | 46 | 156.7 | 36 |
| 59.5 | 40 | 157.0 | 35 |
| 66.9 | 25 | 157.5 | 26 |

† indicates peaks attributed to trapped solvent molecules

### Example 3

### Preparation of Crystalline PF-07104091 Monohydrate (Form 3)

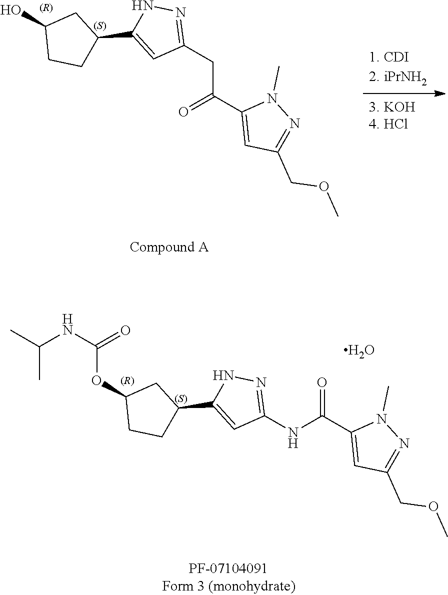

N-(5-((1S,3R)-3-hydroxycyclopentyl)-1H-pyrazol-3-yl)-3-(methoxymethyl)-1-methyl-1H-pyrazole-5-carboxam ide (Compound A) (187 g, 0.586 moles) (prepared by acidic deprotection of intermediate 13B described in Example 13 of U.S. Pat. No. 11,014,911) was dissolved in tetrahydrofuran (THF) (1.78 L) using magnetic stirring with high agitation. The solution was warmed to 25° C., and then 1,1′-carbonyldiimidazole (CDI) (142 g, 0.876 moles) was added. The mixture was stirred at 25° C. for 5 minutes, then warmed to 50° C. at 1° C./min and held for 30 minutes. The mixture was cooled to 30° C., and then charged with 2-propylamine (iPrNH₂) (70 g, 1.184 moles). The reaction was warmed to 50° C. at 1° C./min, and then held at 50° C. until the reaction was complete. If necessary, additional iPrNH₂ (18 g, 0.304 moles) was added to reach completion. Water (1.22 L) was added, and the mixture was warmed to 50° C. A solution of potassium hydroxide (20 g, 0.356 moles) in water (0.561 L) was added, and the mixture was held at 50° C. for 5 hours. The reaction was cooled to 25° C. and the reaction mixture was distilled under vacuum until the solution volume was 6 mL/g based on Compound A, the internal temperature was between 45-50° C., and the THF was less than 0.1% by GCHS. The mixture was cooled to 25° C., then H₂O was added until the reaction volume was 16 mL/g based on Compound A. Acetonitrile (0.75 L) was added via dropping funnel, held for 10 minutes, and then 37% hydrochloric acid in water (29 g) was added over 30 minutes to adjust pH to between 7-8. Additional HCl or KOH was added to adjust the mixture to pH 7-8. The mixture was warmed to 40° C. and held for 3 hours. The mixture was cooled to 15° C. at 0.1° C./min and stirred at 15° C. for 1 hour. The mixture was filtered, and the solids were rinsed with 9:1 H₂O/acetonitrile (0.500 L). The solids were dried in a humidified vacuum oven at 50° C. until Karl Fischer (KF) titration was between 4.2% to 4.5%, to provide PF-07104091 monohydrate (Form 3).

TABLE 4
PXRD peak list for PF-07104091 monohydrate (Form 3)

| Angle (2-theta °) | Relative | Angle (2 theta °) | Relative |
|---|---|---|---|
| ±0.2 °2θ | Intensity (%) | ±0.2 °2θ | Intensity (%) |
| 8.4 | 16.6 | 22.7 | 9.6 |
| 10.1 | 23.4 | 23.1 | 11.6 |
| 11.9 | 10.1 | 23.3 | 5.8 |
| 12.7 | 14.7 | 23.7 | 7.1 |
| 14.4 | 9.1 | 25.4 | 13.1 |
| 16.2 | 13.2 | 25.5 | 16.8 |
| 16.9 | 58.5 | 25.8 | 7.5 |
| 19.2 | 4.3 | 26.8 | 21.0 |
| 19.7 | 10.8 | 27.0 | 31.0 |
| 20.0 | 13.2 | 27.9 | 5.3 |
| 20.3 | 4.0 | 28.6 | 14.1 |
| 20.6 | 7.0 | 29.7 | 3.5 |
| 20.8 | 14.8 | 31.0 | 8.8 |
| 21.5 | 100.0 | 33.6 | 3.4 |
| 21.7 | 15.8 | 34.1 | 7.5 |
| 22.3 | 13.0 | 36.9 | 4.7 |

TABLE 5
Raman peak list for PF-07104091 monohydrate (Form 3)

| Peak position | | Peak position | |
|---|---|---|---|
| cm⁻¹ | Normalized | cm⁻¹ | Normalized |
| ±2 cm⁻¹ | intensity | ±2 cm⁻¹ | intensity |
| 100 | 0.88 | 1107 | 0.13 |
| 163 | 0.31 | 1134 | 0.10 |
| 204 | 0.21 | 1170 | 0.09 |
| 244 | 0.19 | 1180 | 0.14 |
| 300 | 0.14 | 1207 | 0.07 |
| 312 | 0.14 | 1247 | 0.21 |
| 339 | 0.11 | 1272 | 0.58 |
| 368 | 0.10 | 1279 | 0.40 |
| 398 | 0.12 | 1295 | 0.38 |
| 421 | 0.09 | 1313 | 0.20 |
| 449 | 0.14 | 1359 | 0.42 |
| 469 | 0.11 | 1408 | 1.00 |
| 519 | 0.08 | 1448 | 0.72 |
| 568 | 0.12 | 1459 | 0.69 |
| 616 | 0.06 | 1488 | 0.77 |
| 656 | 0.11 | 1556 | 0.32 |
| 717 | 0.12 | 1595 | 0.32 |
| 728 | 0.14 | 1628 | 0.20 |
| 762 | 0.12 | 1657 | 0.92 |
| 785 | 0.09 | 2722 | 0.05 |
| 793 | 0.10 | 2765 | 0.04 |
| 832 | 0.09 | 2813 | 0.13 |
| 846 | 0.09 | 2876 | 0.25 |
| 897 | 0.11 | 2918 | 0.35 |
| 923 | 0.24 | 2943 | 0.41 |
| 955 | 0.18 | 2971 | 0.40 |
| 986 | 0.08 | 2982 | 0.48 |
| 1003 | 0.28 | 3001 | 0.20 |
| 1033 | 0.28 | 3142 | 0.17 |
| 1046 | 0.14 | 3220 | 0.06 |
| 1052 | 0.15 | 3456 | 0.02 |
| 1080 | 0.12 | 3513 | 0.02 |

TABLE 6
¹³C ssNMR peak list for PF-07104091 monohydrate (Form 3)

| ¹³C Chemical | Relative | ¹³C Chemical | Relative |
|---|---|---|---|
| Shifts ppm | Intensity | Shifts ppm | Intensity |
| ±0.2 ppm | (%) | ±0.2 ppm | (%) |
| 23.6 | 56 | 76.6 | 32 |
| 25.2 | 62 | 80.0 | 31 |
| 34.2 | 32 | 92.9 | 43 |
| 35.5 | 100 | 110.9 | 26 |
| 37.5 | 55 | 111.6 | 26 |
| 39.3 | 27 | 135.8 | 36 |
| 40.8 | 27 | 149.2 | 54 |
| 43.9 | 55 | 151.9 | 27 |
| 58.2 | 40 | 152.5 | 26 |
| 58.6 | 41 | 156.7 | 36 |
| 67.8 | 52 | 159.3 | 30 |

### Example 4

### Preparation of Anhydrous Crystalline PF-07104091 (Form 5)

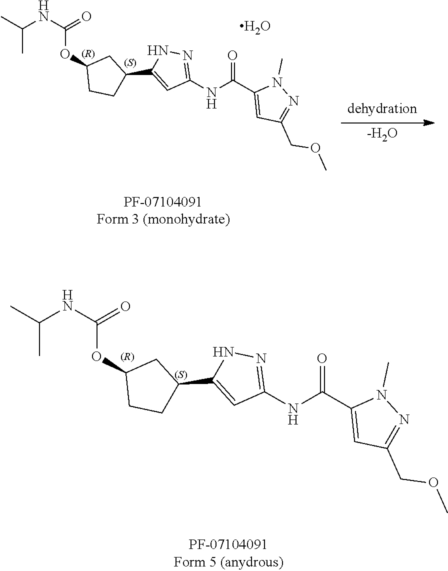

Anhydrous crystalline PF-07104091 (Form 5) was prepared by placing PF-07104091 monohydrate (Form 3) in an open dish within an oven at approximately 50° C. and purging with dry nitrogen gas for approximately 3 hours.

Alternatively, anhydrous crystalline PF-07104091 (Form 5) was prepared by storing PF-07104091 monohydrate (Form 3) at ambient temperature over DRIERITE dessicant (˜0% RH) for 17 days.

Elemental analysis of PF-07104091 (Form 5) was consistent with an anhydrous form, as shown in Table 7.

TABLE 7
Elemental analysis of PF-07104091 (Form 5)

| | Theoretical | Average | Difference from |
|---|---|---|---|
| | anhydrous | (wt %) | Experimental |
| Element | (wt %) | n = 3 | (wt %) |
| Carbon | 56.42 | 56.17 | 0.25 |
| Hydrogen | 6.98 | 6.84 | 0.13 |
| Nitrogen | 20.78 | 20.76 | 0.02 |

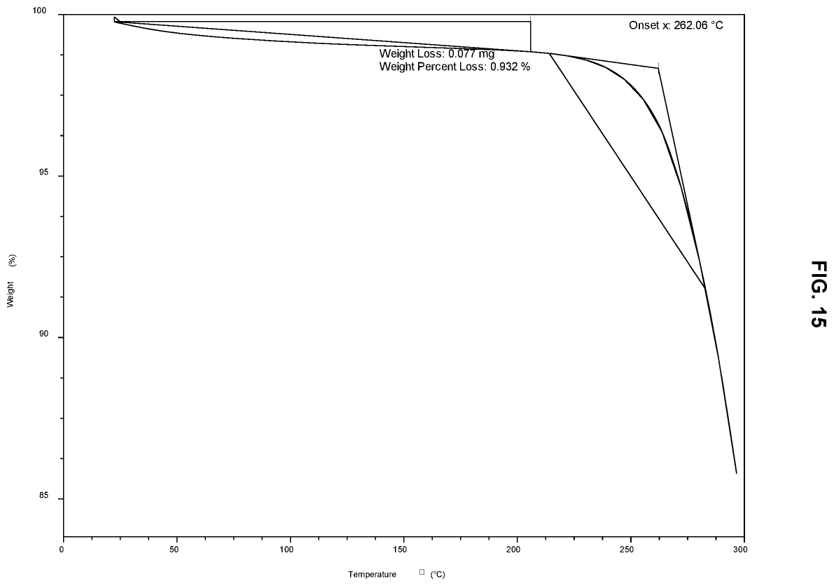

A weight loss of less than 1% at 200° C. was observed by thermogravimetric analysis for PF-07104091 (Form 5), as shown in FIG. 15, confirming the form was anhydrous.

TABLE 8
PXRD peak list for PF-07104091 (Form 5)

| Angle | Relative | Angle | Relative |
|---|---|---|---|
| (2-theta °) | Intensity | (2 theta °) | Intensity |
| ±0.2 °2θ | (%) | ±0.2 °2θ | (%) |
| 10.2 | 29.2 | 19.8 | 100.0 |
| 12.4 | 6.1 | 21.6 | 13.0 |
| 15.4 | 4.7 | 22.5 | 8.6 |
| 17.2 | 5.1 | 23.7 | 3.5 |
| 17.9 | 15.8 | 26.2 | 10.5 |

TABLE 9
Raman peak list for PF-07104091 (Form 5)

| Peak position | | Peak position | |
|---|---|---|---|
| cm⁻¹ | Normalized | cm⁻¹ | Normalized |
| ±2 cm⁻¹ | intensity | ±2 cm⁻¹ | intensity |
| 99 | 0.63 | 1089 | 0.13 |
| 163 | 0.22 | 1130 | 0.08 |
| 198 | 0.19 | 1159 | 0.10 |
| 241 | 0.18 | 1174 | 0.10 |
| 330 | 0.12 | 1208 | 0.06 |
| 374 | 0.13 | 1267 | 0.37 |
| 421 | 0.09 | 1282 | 0.18 |
| 449 | 0.10 | 1302 | 0.16 |
| 467 | 0.09 | 1317 | 0.12 |
| 553 | 0.07 | 1359 | 0.25 |
| 583 | 0.07 | 1378 | 0.43 |
| 641 | 0.10 | 1399 | 0.58 |
| 670 | 0.08 | 1413 | 0.36 |
| 717 | 0.09 | 1445 | 0.54 |
| 734 | 0.11 | 1485 | 0.57 |
| 766 | 0.07 | 1551 | 0.29 |
| 785 | 0.06 | 1591 | 0.36 |
| 827 | 0.06 | 1668 | 1.00 |
| 850 | 0.09 | 2722 | 0.03 |
| 896 | 0.19 | 2823 | 0.12 |
| 910 | 0.19 | 2877 | 0.14 |
| 980 | 0.08 | 2941 | 0.38 |
| 1003 | 0.19 | 2968 | 0.39 |
| 1018 | 0.12 | 3132 | 0.07 |
| 1025 | 0.13 | 3192 | 0.05 |
| 1048 | 0.21 | 3268 | 0.05 |
| 1075 | 0.12 | | |

TABLE 10
¹³C ssNMR peak list for PF-07104091 (Form 5)

| ¹³C Chemical | Relative | ¹³C Chemical | Relative |
|---|---|---|---|
| Shifts ppm | Intensity | Shifts ppm | Intensity |
| ±0.2 ppm | (%) | ±0.2 ppm | (%) |
| 21.6 | 35 | 78.7 | 62 |
| 22.7 | 53 | 94.4 | 53 |
| 23.1 | 58 | 109.9 | 41 |
| 27.0 | 28 | 135.3 | 42 |
| 33.4 | 64 | 144.6 | 23 |
| 34.6 | 62 | 146.9 | 27 |
| 35.6 | 100 | 149.5 | 47 |
| 40.8 | 99 | 152.3 | 37 |
| 43.5 | 80 | 153.6 | 32 |
| 57.0 | 66 | 157.2 | 61 |
| 67.7 | 30 | 158.1 | 62 |
| 69.0 | 40 | | |

### Example 5

### Preparation of Amorphous PF-07104091 (Form 4)

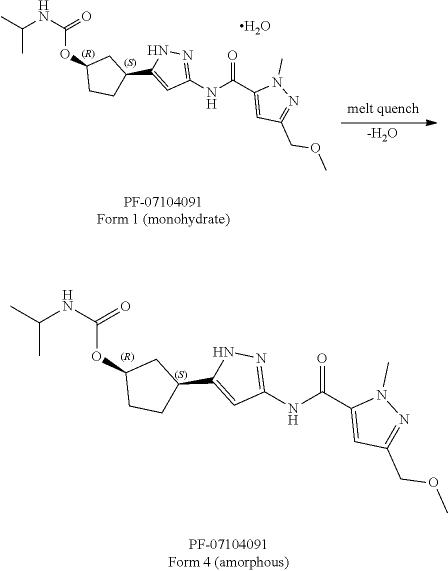

Amorphous PF-07104091 (Form 4) was prepared using in-situ melt quenching of PF-07104091 monohydrate (Form 1) (prepared as described in U.S. Pat. No. 11,014,911) within a differential scanning calorimeter (DSC). Larger scale preparations of the amorphous Form 4 were attempted using both melt quenching and lyophilization.

The general DSC procedure used to provide amorphous PF-07104091 (Form 4) is provided below:
1. Weigh 3-5 mg of API into an aluminum pan and seal non-hermetically with aluminum lid
2. Load pan into DSC under 50 L/min nitrogen gas purge
3. Ramp to 20° C./min to -30° C.
4. Hold isothermal 1 minute
5. Ramp 10° C./min to 160° C.
6. Hold isothermal 1 minute
7. Ramp 20° C./min tot -30° C.
8. Hold isothermal 1 minute
9. Ramp 10° C./min to 160° C.

A representative DSC thermogram is provided in FIG. 14 from the second heating cycle (i.e., step 9 above), showing a glass transition temperature (T_{g}) of about 59.8 ±5° C. (as measured by DSC at a ramp rate of 10° C./min).

Amorphous PF-07104091 (Form 4) has a PXRD pattern (2θ) comprising a broad peak at diffraction angles (20) from about 5 to about 35 °2θ±0.2 °2θ, without any of the sharp peaks characteristic of a crystalline form, as shown in FIG. 4.

### Comparative Example 6

### Crystalline PF-07104091 Monohydrate (Form 1)

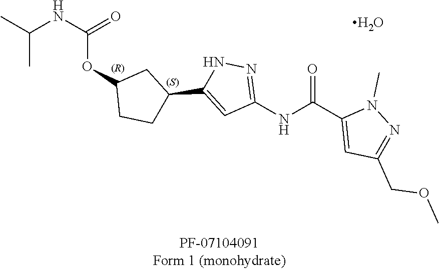

PF-07104091 monohydrate (Form 1) was prepared as described in Example 13 of U.S. Pat. No. 11,014,911. PXRD, Raman, and ¹³C ssNMR characterization data for Form 1 are provided in Tables 11, 12 and 13, respectively.

TABLE 11
PXRD peak list for PF-07104091 monohydrate (Form 1)

| Angle | Relative | Angle | Relative |
|---|---|---|---|
| (2-theta °) | Intensity | (2 theta °) | Intensity |
| ±0.2 °2θ | (%) | ±0.2 °2θ | (%) |
| 3.9 | 19.5 | 25.0 | 25.9 |
| 9.1 | 18.3 | 25.7 | 8.3 |
| 10.4 | 96.5 | 26.0 | 10.1 |
| 11.7 | 64.3 | 26.3 | 15.1 |
| 12.9 | 41.4 | 26.6 | 8.4 |
| 16.0 | 15.5 | 27.0 | 5.0 |
| 18.2 | 100.0 | 27.6 | 21.3 |
| 18.6 | 14.4 | 28.2 | 31.7 |
| 19.4 | 38.1 | 28.9 | 5.2 |
| 19.6 | 20.3 | 30.4 | 6.8 |
| 20.0 | 10.5 | 31.1 | 7.8 |
| 20.3 | 20.6 | 31.5 | 9.9 |
| 20.6 | 43.0 | 33.9 | 11.6 |
| 20.8 | 26.1 | 35.1 | 3.3 |
| 21.0 | 23.7 | 35.8 | 3.0 |
| 22.2 | 20.6 | 36.6 | 7.1 |
| 22.7 | 3.4 | 37.6 | 3.9 |
| 23.5 | 22.9 | 38.3 | 5.2 |
| 24.2 | 64.0 | | |

TABLE 12
Raman peak list for PF-07104091 monohydrate (Form 1)

| Peak position | | Peak position | |
|---|---|---|---|
| cm⁻¹ | Normalized | cm⁻¹ | Normalized |
| ±2 cm⁻¹ | intensity | ±2 cm⁻¹ | intensity |
| 107 | 0.38 | 1074 | 0.11 |
| 134 | 0.21 | 1084 | 0.09 |
| 150 | 0.18 | 1103 | 0.05 |
| 163 | 0.30 | 1134 | 0.04 |
| 174 | 0.13 | 1155 | 0.07 |
| 179 | 0.14 | 1168 | 0.09 |
| 200 | 0.12 | 1185 | 0.08 |
| 263 | 0.08 | 1242 | 0.17 |
| 311 | 0.09 | 1264 | 0.15 |
| 337 | 0.08 | 1274 | 0.16 |
| 351 | 0.14 | 1294 | 0.14 |
| 375 | 0.06 | 1303 | 0.12 |
| 421 | 0.08 | 1327 | 0.07 |
| 432 | 0.07 | 1348 | 0.21 |
| 449 | 0.12 | 1386 | 0.31 |
| 497 | 0.05 | 1402 | 0.42 |
| 523 | 0.06 | 1420 | 0.21 |
| 537 | 0.05 | 1449 | 0.32 |
| 616 | 0.04 | 1463 | 0.40 |
| 645 | 0.07 | 1470 | 0.43 |
| 662 | 0.05 | 1484 | 0.39 |
| 701 | 0.04 | 1494 | 0.16 |
| 717 | 0.10 | 1552 | 0.44 |
| 758 | 0.06 | 1585 | 0.26 |
| 785 | 0.05 | 1683 | 1.00 |
| 817 | 0.05 | 2832 | 0.10 |
| 853 | 0.04 | 2870 | 0.14 |
| 872 | 0.16 | 2879 | 0.15 |
| 890 | 0.06 | 2902 | 0.28 |
| 896 | 0.07 | 2939 | 0.36 |
| 905 | 0.16 | 2960 | 0.32 |
| 932 | 0.08 | 2979 | 0.29 |
| 942 | 0.06 | 2996 | 0.16 |
| 956 | 0.05 | 3131 | 0.06 |
| 981 | 0.06 | 3228 | 0.04 |
| 1000 | 0.21 | 3244 | 0.04 |
| 1027 | 0.08 | 3278 | 0.04 |
| 1041 | 0.12 | 3299 | 0.05 |
| 1060 | 0.07 | 3336 | 0.07 |

TABLE 13
¹³C ssNMR peak list for PF-07104091 monohydrate (Form 1)

| ¹³C Chemical | Relative | ¹³C Chemical | Relative |
|---|---|---|---|
| Shifts ppm | Intensity | Shifts ppm | Intensity |
| ±0.2 ppm | (%) | ±0.2 ppm | (%) |
| 22.4 | 58 | 66.6 | 61 |
| 22.9 | 60 | 73.8 | 45 |
| 24.5 | 52 | 76.5 | 45 |
| 26.0 | 48 | 94.3 | 40 |
| 29.1 | 39 | 94.9 | 40 |
| 32.2 | 36 | 104.2 | 61 |
| 35.4 | 51 | 137.1 | 48 |
| 36.4 | 94 | 147.0 | 56 |
| 36.9 | 63 | 148.4 | 52 |
| 39.1 | 42 | 149.6 | 46 |
| 40.3 | 77 | 149.9 | 44 |
| 43.5 | 72 | 156.3 | 100 |
| 56.7 | 59 | | |

### Example 7

### Stability Studies

Slurry experiments were conducted as follows. The indicated starting form of PF-07104091 was transferred to a 2 mL vial. The indicated solvent or solvent mixture was added to obtain a slurry at the specified temperature (Table 14). Additional solids were added as necessary to ensure a sufficiently thick slurry. A magnetic stir bar was added and the vial was tightly capped to prevent solvent loss. The resulting slurry was left to stir at the specified temperature. Aliquots were pulled from the slurry periodically or after a certain duration. Solids were separated from the liquid by centrifuge filtration and the solids were characterized by powder x-ray diffraction. PF-07104091 monohydrate (Form 3) was the thermodynamically most stable form at 4° C., ˜25° C. (ambient), and 40° C.

TABLE 14
Stability Experiments

| | Temperature | | |
|---|---|---|---|
| Solvent System | (° C.) | Starting Form | Final Form |
| 2-propanol/water, Aw 0.9 | 4 | Form 1 | Form 3 |
| 2-propanol/water, Aw 0.9 | ambient | Form 1 | Form 3 |
| 2:3 v/v DMA/water | ambient | Form 1 & Form 3 | Form 3 |
| 1:1 v/v 2-propanol/water | ambient | Form 1 & Form 3 | Form 3 |
| 1:3 v/v 2-propanol/water | ambient | Form 1 & Form 3 | Form 3 |
| 1:9 v/v 2-propanol/water | ambient | Form 1 & Form 3 | Form 3 |
| 1:1 v/v acetonitrile/water | ambient | Form 1 & Form 3 | Form 3 |
| 1:4 v/v acetonitrile/water | ambient | Form 1 & Form 3 | Form 3 |
| Water | 40 | Form 1 & Form 3 | Form 3 |
| 2:3 v/v DMA/water | 40 | Form 1 & Form 3 | Form 3 |
| 1:3 v/v 2-propanol/water | 40 | Form 1 & Form 3 | Form 3 |
| 1:9 v/v 2-propanol/water | 40 | Form 1 & Form 3 | Form 3 |

Note:
Aw = water activity
v/v = volume/volume

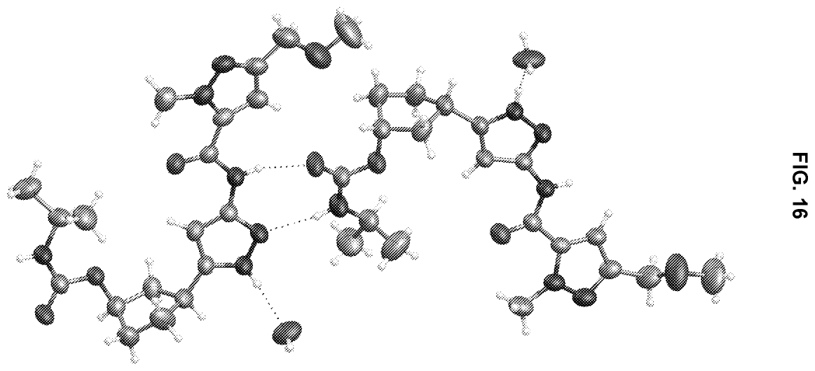

A single crystal x-ray structure of PF-07104091 monohydrate (Form 3) was determined and is shown in FIG. 16. Computational analyses showed that PF-07104091 monohydrate (Form 3) had a superior intermolecular geometry, hydrogen-bonding network topology, and lack of void space relative to PF-07104091 monohydrate (Form 1), and was therefore expected to be more stable. The single crystal x-ray structure of PF-07104091 monohydrate (Form 1) was provided in FIG. 1 of U.S. Pat. No. 11,014,911.

1. A crystalline form of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (PF-07104091) monohydrate (Form 3), having a powder X-ray diffraction (PXRD) pattern comprising peaks at 2θ values measured using CuKα radiation of: 8.4, 10.1 and 21.5 °2θ±0.2 °2θ.

2. The crystalline form of claim 1, having a PXRD pattern further comprising a peak at a 2θ value measured using CuKα radiation of: 16.9 °2θ±0.2 °2θ.

3. The crystalline form of claim 1 or 2, having a PXRD pattern further comprising a peak at a 2θ value measured using CuKα radiation of: 27.0 °2θ±0.2 °2θ.

4. The crystalline form of any one of claims 1 to 3, having a Raman spectrum comprising one, two or three wavenumber (cm⁻¹) values selected from the group consisting of: 1657, 1595 and 1408 cm⁻¹+2 cm⁻¹.

5. The crystalline form of any one of claims 1 to 4, having a ¹³C solid state NMR spectrum comprising one, two or three resonance (ppm) values selected from the group consisting of: 25.2, 37.5 and 159.3 ppm±0.2 ppm.

6. The crystalline form of claim 5, having a ¹³C solid state NMR spectrum further comprising resonance (ppm) values of: 151.9 and 152.5 ppm±0.2 ppm.

7. A crystalline form of PF-07104091 monohydrate (Form 3), having a ¹³ C solid state NMR spectrum comprising resonance (ppm) values of: 25.2, 37.5 and 159.3 ppm±0.2 ppm.

8. The crystalline form of claim 7, having a ¹³C solid state NMR spectrum further comprising one or two resonance (ppm) values selected from the group consisting of: 151.9 and 152.5 ppm±0.2 ppm.

9. The crystalline form of claim 7 or 8, having a PXRD pattern comprising peaks at 2θ values measured using CuKα radiation of: 8.4 and 10.1 °2θ±0.2 °2θ.

10. The crystalline form of any one of claims 1 to 9, wherein the crystalline form is substantially pure PF-07104091 monohydrate (Form 3).

11. An anhydrous crystalline form of PF-07104091 (Form 2), having a PXRD pattern comprising peaks at 2θ values measured using CuKα radiation of: 9.8, 13.3 and 7.4 °2θ±0.2 °2θ.

12. The crystalline form of claim 11, having a PXRD pattern further comprising a peak at a 2θ value measured using CuKα radiation of: 4.2 °2θ±0.2 °2θ.

13. The crystalline form of claim 11 or 12, having a PXRD pattern further comprising a peak at a 2θ value measured using CuKα radiation of: 7.5 °2θ±0.2 °2θ.

14. The crystalline form of any one of claims 11 to 13, having a Raman spectrum comprising one, two or three wavenumber (cm⁻¹) values selected from the group consisting of: 1691, 1582 and 996 cm⁻¹±2 cm⁻¹.

15. The crystalline form of any one of claims 11 to 14, having a ¹³C solid state NMR spectrum comprising one, two or three resonance (ppm) values selected from the group consisting of: 24.1, 39.8 and 41.6 ppm±0.2 ppm.

16. The crystalline form of claim 15, having a ¹³C solid state NMR spectrum further comprising resonance (ppm) values of: 21.8 and 138.2 ppm±0.2 ppm.

17. The crystalline form of any one of claims 11 to 16, wherein the crystalline form is substantially pure PF-07104091 (Form 2).

18. An anhydrous crystalline form of PF-07104091 (Form 5), having a PXRD pattern comprising three or more peaks at 2θ values measured using CuKα radiation selected from the group consisting of: 10.2, 12.4, 15.4, 17.2, 17.9, 19.8, 21.6, 22.5, 23.7 and 26.2 °2θ±0.2 °2θ.

19. A pharmaceutical composition comprising the crystalline form of any one of claims 1 to 18, and a pharmaceutically acceptable carrier or excipient.

20. An amorphous form of PF-07104091 (Form 4), having a PXRD pattern comprising a broad peak at diffraction angles (20) measured using CuKα radiation from about 5 to about 35 °2θ±0.2 °2θ.

21. The amorphous form of claim 20, having a PXRD pattern essentially the same as in FIG. 4.

22. The amorphous form of claim 20 or 21, having a glass transition temperature (Tg) of 59.8 ±5° C.

23. A pharmaceutical composition comprising the amorphous form of any one of claims 20 to 22, and a pharmaceutically acceptable carrier or excipient.

24. A method of treating cancer in a subject in need thereof, comprising administering to the subject a therapeutically effective amount of the crystalline form of any one of claims 1 to 18 or the amorphous form of any one of claims 20 to 22.

25. The method of claim 24, wherein the cancer is selected from the group consisting of breast cancer, prostate cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.

26. The method of claim 24 or 25, further comprising administering to the subject an amount of an additional anticancer agent.
